(19) United States
(12) Patent Application Publication
(10) Pub. No.: US 2026/0263611 A1
(43) Pub. Date: Sep. 10, 2026

(54) Antibiotic Vancomycin-Teixobactin Conjugates

(71) Applicant: The Regents of the University of California, Oakland, CA (US)

(72) Inventors: Maria Sophia Teresa L. Padilla, Irvine, CA (US); James S. Nowick, Irvine, CA (US)

(73) Assignee: The Regents of the University of California, Oakland, CA (US)

(21) Appl. No.: 19/534,675

(22) Filed: Feb. 9, 2026

Related U.S. Application Data
(60) Provisional application No. 63/756,041, filed on Feb. 7, 2025.

(51) Int. Cl. A61K 47/64; C07K 9/00; C07K 11/00

(52) U.S. Cl. CPC A61K 47/646; C07K 9/008; C07K 11/00; C07K 2319/00

(57) ABSTRACT

Vancomycin-teixobactin conjugates, methods of their synthesis, and methods of their use as antimicrobial treatments are described. The vancomycin-teixobactin conjugates have synergistic activity and overcome issues of antibiotic resistance. The vancomycin-teixobactin conjugates can be utilized to treat infections of antibiotic-resistant bacteria.

### CROSS REFERENCE TO RELATED APPLICATIONS

The current application claims the benefit of U.S. Provisional Application No. 63/756,041, entitled “Antibiotic Vancomycin-Teixobactin Conjugates”, filed Feb. 7, 2025, the disclosure of which is hereby incorporated by reference in its entirety.

### TECHNICAL FIELD

The present disclosure is related to antibiotic conjugates, methods of synthesis and methods of treatment; and more particularly to conjugates of vancomycin with teixobactin.

### BACKGROUND

Antimicrobial agents, including antibiotics, are used for the treatment or prevention of pathogenic bacterial infections. A major complication in the medical and health field, however, is antimicrobial resistance by pathogenic bacteria. The paucity of novel antimicrobial compounds exacerbates this problem, as treatments continue to utilize classical antibiotics.

Furthermore, antibiotic-resistant bacteria are becoming an even greater concern as the number of deaths due to these infections increases and antibiotic efficacy decreases. Gram-positive pathogens—including methicillin-resistant Staphylococcus aureus (MRSA), vancomycin-resistant Enterococci (VRE), and multidrug-resistant Mycobacterium tuberculosis (MDR-TB)—cause more than 45% of the deaths related to antibiotic resistant infections in the United States and more than 35% worldwide.

In 2015, Lewis and coworkers discovered teixobactin (FIG. 1; SEQ ID NO: 1), a non-ribosomal undecapeptide consisting of a linear tail and a macrolactone ring (L. L. Ling, et al., Nature 2015; 517:455-459, the disclosure of which is incorporated herein by reference.). Teixobactin inhibits gram-positive bacteria—including MRSA, VRE, and MDR-TB—by restricting peptidoglycan cell wall synthesis, a critical component for pathogen survival. As of yet, teixobactin resistant strains have not yet emerged due to their mechanism of inhibition that attacks the cell wall component, which is integral and conserved across all Gram-positive bacteria.

### SUMMARY

Embodiments of the disclosure are directed to antimicrobial vancomycin-teixobactin conjugates, methods of their synthesis, and methods of their administration. In several embodiments, a vancomycin-teixobactin conjugate comprises a teixobactin linked with vancomycin. In various embodiments, teixobactin and/or vancomycin is modified from their naturally occurring structure. In many embodiments, a vancomycin-teixobactin conjugate is administered to a subject as an antibiotic treatment, in which conjugation provides enhanced and synergistic antibiotic activity. Accordingly, in many embodiments, a vancomycin-teixobactin conjugate is administered to treat the subject for an infection or Gram-positive bacteria or prophylactically administered to prevent infection of Gram-positive bacteria.

### BRIEF DESCRIPTION OF THE DRAWINGS

These and other features and advantages of the present invention will be better understood by reference to the following detailed description when considered in conjunction with the accompanying data and figures, wherein:

FIG. 1A provides a chemical structure diagram of teixobactin.

FIG. 1B provides a chemical structure diagram of vancomycin.

FIG. 2 provides a schematic of a design for a vancomycin-teixobactin conjugate.

FIGS. 3A and 3B provide chemical structure diagrams of teixobactin rings and teixobactin ring analogues.

FIG. 4 provides a chemical structure diagram of a teixobactin ring showing linkage to a teixobactin tail.

FIGS. 5A to 5H provide chemical structure diagrams of teixobactin tails and teixobactin analogues.

FIGS. 6A to 6G provide chemical structure diagrams of compounds that can be optionally linked to the N-terminus of a teixobactin tail.

FIGS. 7A to 7D provide chemical structure diagrams of teixobactin compounds and sites for linkage to vancomycin.

FIGS. 8A to 8G provide chemical structure diagrams of linkers that can be used to conjugate vancomycin with a teixobactin ring.

FIG. 9 provides a Markush chemical structure vancomycin and vancomycin analogues.

FIGS. 10A to 10Z provide chemical structures of vancomycin-teixobactin conjugates and their MIC values against various Gram-positive bacteria.

FIG. 11 provides pharmacokinetic data comparing a vancomycin-teixobactin conjugate and vancomycin.

FIGS. 12A and 12B provide data demonstrating antibiotic activity of a vancomycin-teixobactin conjugate administered to neutropenic mice infected with gram-positive bacteria.

FIGS. 13A to 13B provide data demonstrating antibiotic activity of a vancomycin-teixobactin conjugate administered to neutropenic mice infected with difficult-to-treat gram-positive bacteria.

FIG. 13C provides data demonstrating the inability of vancomycin to adequately treat neutropenic mice infected with difficult-to-treat gram-positive bacteria.

FIG. 14 provides a representative synthetic scheme of Lys₁₀-teixobactin and other analogues.

FIG. 15 provides data on MIC values of vancomycin and teixobactin derivatives.

FIG. 16 provides chemical structures of truncated Lys₁₀-teixobactin analogues.

FIG. 17 provides data on MIC values of vancomycin and truncated teixobactin derivatives and conjugates.

FIG. 18 provides data results of a time kill assay of Cbp-Lys₁₀-teixo₇₋₁₁-vanco and vancomycin at 1 μg/mL against MRSA cells. This concentration is 2× the MIC of Cbp-Lys₁₀-teixo₇₋₁₁-vanco against MRSA.

### DETAILED DISCLOSURE

The disclosure provides description and figures related to vancomycin-teixobactin conjugates, including chemical compounds, medicinal formulations, methods of their synthesis, and applications of their use such as antimicrobial treatments. In several embodiments, a compound comprises a vancomycin and teixobactin conjugated together. In many embodiments, the teixobactin of the vancomycin-teixobactin conjugate comprises a teixobactin ring. In several embodiments, the vancomycin of the vancomycin-teixobactin conjugate comprises vancomycin or an analogue of vancomycin. Several of the described conjugates provide enhanced properties including improved antimicrobial activity as compared to either compound alone or to mixtures of the two compounds together. These properties yield next-generation medicinal formulations for treatment of gram-positive bacterial infections, including antibiotic-resistant bacteria. As such, the various compounds of the disclosure enable much-needed advancement for the treatment of patients suffering from difficult-to-treat infections.

In several embodiments, a vancomycin-teixobactin conjugate comprises a teixobactin ring inclusive of residue positions eight to eleven. In many embodiments, the teixobactin ring is linked with a tail that extends from the N-terminus the amino acid of residue position 8. The teixobactin-ring tail can include more residues of teixobactin, including up to all seven residue positions (e.g., teixobactin₁₋₇). In some embodiments, a teixobactin-ring tail comprises one or more chemical groups, which can extend from or replace one or more residues of the teixobactin. In some embodiments, a chemical moiety is linked to the N-terminus of a teixobactin amino acid.

In many embodiments, a vancomycin-teixobactin conjugate comprises a vancomycin conjugated to a teixobactin ring. In various embodiments, natural vancomycin or an analogue thereof is utilized. In some embodiments, conjugation between teixobactin comprises a linker is utilized between an amino acid of teixobactin and vancomycin, which can provide a distance and/or flexibility between the teixobactin ring structure and the vancomycin structure. Linkage from teixobactin can be achieved via a free C-terminal group, N-terminal group, or an amino acid side chain. Linkage from vancomycin can be achieved by a free amino group or carboxyl group.

Vancomycin-teixobactin conjugates have antibiotic properties against gram-positive bacteria. In many embodiments, a vancomycin-teixobactin conjugate is formulated into a medicament, which can be utilized as an antibiotic and administered to an individual for treatment of a bacterial infection. Vancomycin is a well-known and useful antibiotic, but several bacterial strains have grown resistance to it. Teixobactin has strong antimicrobial activity against gram-positive bacteria including most (if not all) antibiotic-resistant bacteria but is difficult to administer and has poor pharmacokinetics because the peptide has a propensity to form aggregates that clump and precipitate. In a series of experiments, it was unexpectedly found that conjugating a teixobactin ring to vancomycin results in a compound with high solubility with synergistic antibiotic activity, including antibiotic activity against antibiotic-resistant bacteria. These experiments surprisingly found that vancomycin-teixobactin conjugates have better antibiotic activity than vancomycin alone, teixobactin alone, or mixtures of unconjugated vancomycin with teixobactin. Based on this activity, in accordance with several embodiments, a vancomycin-teixobactin conjugate is formulated and administered to treat an individual with a gram-positive pathogen infection, including (but not limited to) to infections of Staphylococcus, Streptococcus, Clostridium, Bacillus, Corynebacterium, and Listeria. Furthermore, in accordance with many embodiments, a vancomycin-teixobactin conjugate is formulated and administered to treat an individual with an infection of difficult-to-treat pathogens such as (for example) Bacillus anthracis (Anthrax), Staphylococcus aureus (MRSA), vancomycin-resistant Enterococci (VRE), and multidrug-resistant Mycobacterium tuberculosis (MDR-TB).

Throughout the description and claims, the term “residue position” is in reference to the teixobactin peptide as isolated from its natural source. Accordingly, the residue position of various embodiments of teixobactin peptides, teixobactin analogues, and/or enantiomers of teixobactin peptides or teixobactin analogues is based on the position of the natural teixobactin peptide regardless of whether an insertion, deletion or substitution is present in the peptide. For example, various embodiments of a teixobactin ring are described to have an amino acid with and a side chain having an amino terminus at residue position 10, which is a substitution of allo-enduraciddine (allo-END) of the naturally occurring teixobactin peptide. For simplicity and consistency, the various embodiments of peptides having one or more of amino acid substitutions are described and claimed throughout as occurring at residue position as determined by the naturally occurring teixobactin, regardless of whether a particular embodiment includes an insertion, deletion, or substitution that would alter the actual residue position. Furthermore, the description and claims of embodiments of teixobactin can have a particular amino acid at a particular residue position, but does this does not preclude these embodiments from having an insertion, deletion, or substitution. For example, a vancomycin-teixobactin conjugate may be described as having a lysine substituted at residue position 4, but this does not define that this embodiment of the vancomycin-teixobactin conjugate necessarily includes amino acids at residue positions 1-3 or 5-7.

Throughout the description and claims, the term “vancomycin-teixobactin conjugate” is utilized to refer to conjugates comprising a teixocbactin ring (i.e., teixobactin₈₋₁₁) and vancomycin. The term is to include naturally occurring compounds and analogues of vancomycin and teixobactin. An analogue is a modified naturally occurring compound or is a synthesized compound that has a structure to resemble the naturally occurring compound. As is readily appreciated in the field of medicine, several analogues of vancomycin have been developed. Examples of vancomycin analogues can be found described within the literature (See, e.g., J. Chang, et al., ChemMedChem. 2013 June; 8 (6): 976-84; V. Yarlagadda, et al., J Med Chem. 2014 Jun. 12; 57 (11): 4558-68; A. Okano, et al., J Am Chem Soc. 2015 Mar. 18; 137 (10): 3693-704; S. Yoganathan and S. J. Miller, J Med Chem. 2015 Mar. 12; 58 (5): 2367-77; V. Yarlagadda, et al., ACS Infect Dis. 2016 Feb. 12; 2 (2): 132-9; A. Okano, et al., Proc Natl Acad Sci USA. 2017 Jun. 27; 114 (26): E5052-E5061; S. M. Silverman, et al., Chemistry. 2017 Jan. 1; 23 (1): 79-83; A. Okano, et. al., Chem Rev. 2017 Sep. 27; 117 (18): 11952-11993; D. Guan, et al., Angew Chem Int Ed Engl. 2019 May 13; 58 (20): 6678-6682; G. Dhanda, et al., J Med Chem. 2019 Apr. 11; 62 (7): 3184-3205; Z. C. Wu, et al., ACS Infect Dis. 2020 Aug. 14; 6 (8): 2169-2180; E. Muhlberg, et al., Can J Microbiol. 2020 January; 66 (1): 11-16; Z. C. Wu and D. L. Boger, Acc Chem Res. 2020 Nov. 17; 53 (11): 2587-2599; I. S. Shchelik and K. Gademann, ACS Med Chem Lett. 2021 Oct. 18; 12 (12): 1898-1904; Y. Acharya, et al., Chem Commun (Camb). 2022 Feb. 8; 58 (12): 1881-1897; L. Zhai, et al., Molecules. 2022 Nov. 9; 27 (22): 7685; P. Sarkar, et al., Chem Sci. 2023 Jan. 6; 14 (9): 2386-2398; the disclosures of which are each hereby incorporated by reference). Many analogues and/or modified compounds of teixobactin are described herein.

### Vancomycin and Teixobactin Structure and Function

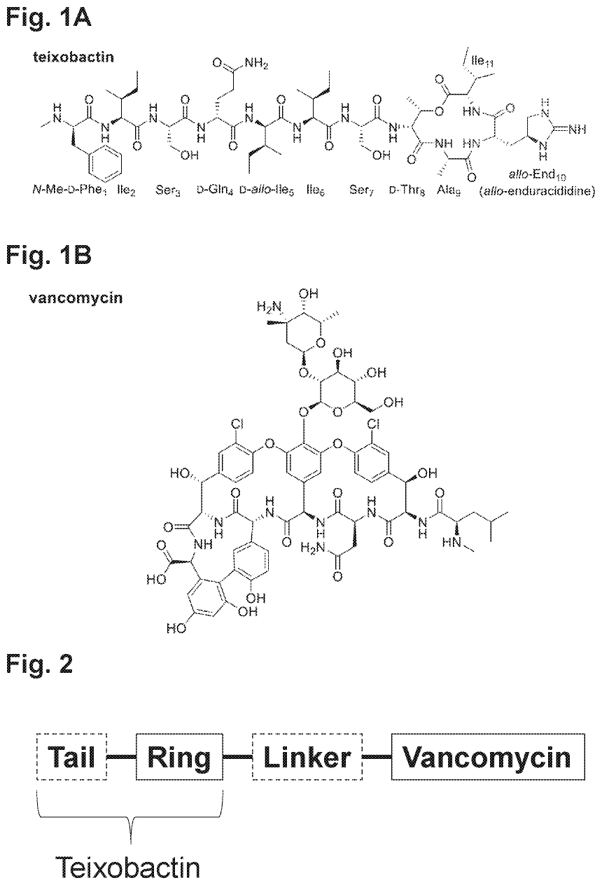

The antibiotic teixobactin, first reported in 2015, is a nonribosomal undecapeptide containing a macrocyclic depsipeptide group (FIG. 1A, SEQ ID NO: 1). As such, it contains four D-amino acids and seven L-amino acids, wherein the C-terminal's Ile₁₁ is cyclized onto the side chain of D-Thr₈ to form a 13-membered lactone. In addition, residue 10 of teixobactin is a rare nonproteinogenic amino acid, L-allo-enduracididine (allo-End₁₀), which is a cyclic analogue of arginine.

Vancomycin is a glycopeptide antibiotic (FIG. 1B) synthesized by soil bacterium Amycolatopsis orientalis. Vancomycin is nonribosomally synthesized to form a linear heptapeptide that comprises several non-proteinogenic amino acids, which is further crosslinked and glycosylated to yield the biologically active molecule.

Vancomycin and teixobactin have a similar mechanism action in which both compounds inhibit cell wall formation in Gram-positive bacteria by binding to the wall's lipid II and related peptidoglycan precursors. Vancomycin and teixobactin target different portions of lipid II. Vancomycin associates with D-Ala-D-Ala of lipid II whereas teixobactin associates with the pyrophosphates of the prenyl-pyrophosphate-GlcNAc region. Bacteria strains can gain resistance to vancomycin by mutating D-Ala-D-Ala of lipid II to D-Ala-D-Lac, negating the ability of vancomycin to associate with the cell wall. The prenyl-pyrophosphate-GlcNAc region is highly conserved in bacteria and cannot easily mutate to impart drug resistance. It was postulated that tethering the vancomycin to the teixobactin, teixobactin's ability to engage with the prenyl-pyrophosphate-GlcNAc region could potentially position vancomycin proximal to the bacterial cell wall such that it can impart antibiosis. Whether vancomycin-teixobactin conjugates would be able to improve upon the antimicrobial activity of vancomycin and teixobactin was not able to be predicted prior to the experiments performed as described herein. It was unknowable whether teixobactin's solubility issues of and/or vancomycin's inability to associate with D-Ala-D-Lac of vancomycin would be solved by conjugation together.

### Vancomycin-Teixobactin Conjugates

Several embodiments are directed to vancomycin-teixobactin conjugates (FIG. 2). A vancomycin-teixobactin conjugate can comprise a teixobactin ring conjugated to vancomycin. In some embodiments, vancomycin and a texiobactin ring are conjugated directly without a linker. One example of a vancomycin-teixobactin conjugate without a linker can comprise a teixobactin ring having an amino acid that linked with a functional group of vancomycin. In some embodiments, vancomycin and a texiobactin ring are conjugated with a linker in between (FIG. 2). In some embodiments, a tail is linked to the N-terminus of the teixobactin ring.

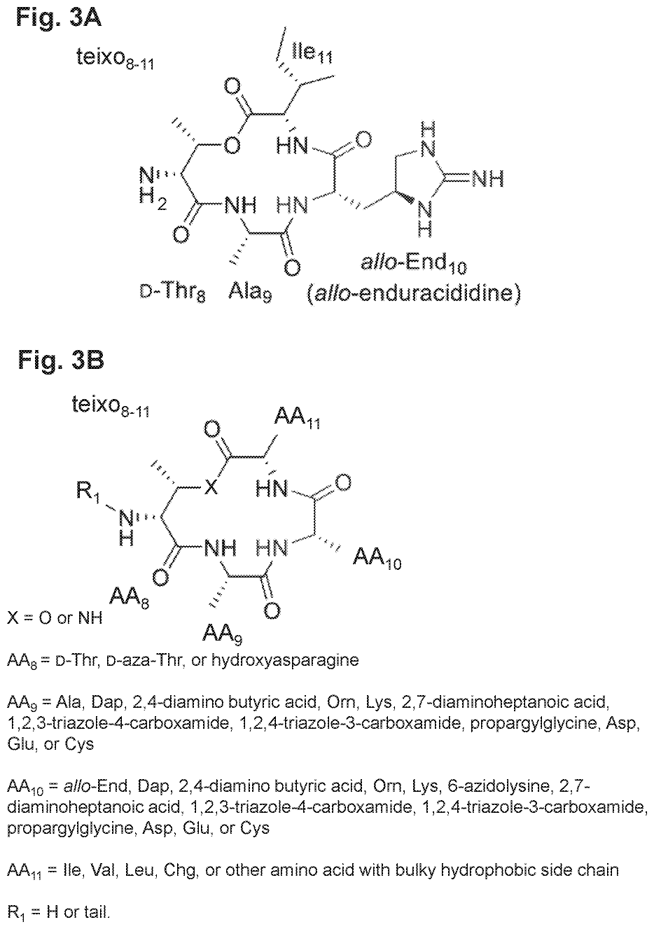

A teixobactin ring comprises amino acids 8-11. The naturally occurring texiobactin ring comprises D-Thr8, Ala9, allo-End₁₀, and Ile₁₁ (FIG. 3A, SEQ ID NO: 2). Several embodiments of a vancomycin-teixobactin conjugate comprise a teixobactin ring with one or more substitutions of naturally occurring teixobactin. Provided in FIG. 3B (SEQ ID NO: 3) is a structure diagram of various teixobactin rings that can be utilized with a vancomycin-teixobactin conjugate in accordance with various embodiments.

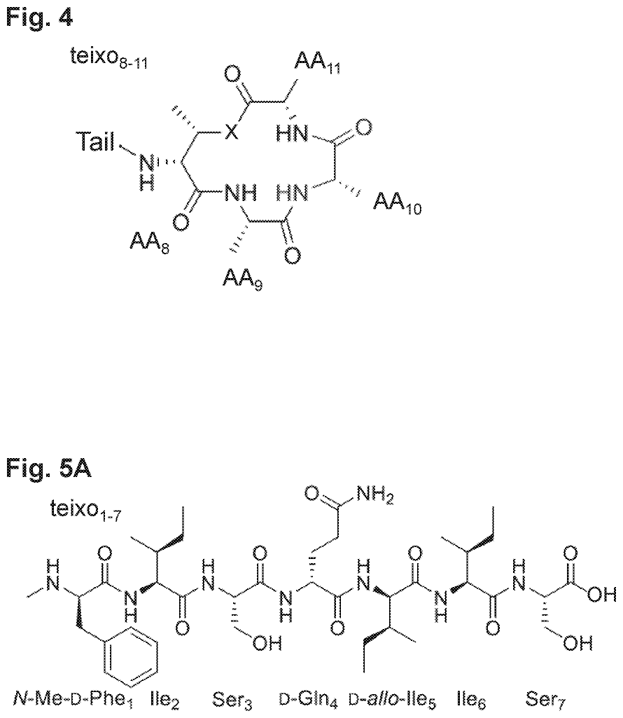

In some embodiments, a teixobactin ring comprises the naturally occurring D-Threonine (D-Thr) at residue position 8 (AA₈). In some embodiments, a teixobactin ring comprises a substituted amino acid at residue position 8. In some embodiments, a teixobactin ring comprises D-aza-Threonine (D-aza-Thr) at residue position 8. In some embodiments, a teixobactin ring comprises hydroxyasparagine at residue position 8. Referring to the structure in FIG. 3B, when AAs is D-Thr, X is O; and when AAs is D-aza-Thr, X is NH. In some embodiments, a tail is linked at the N-terminus of AA₈ (FIG. 4A).

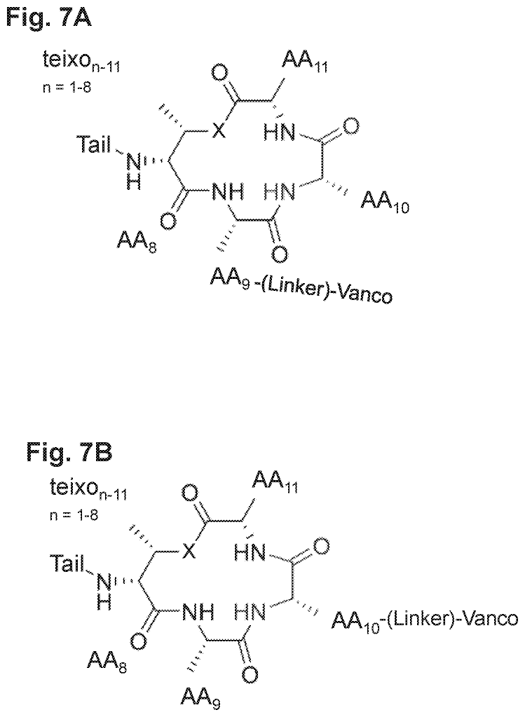

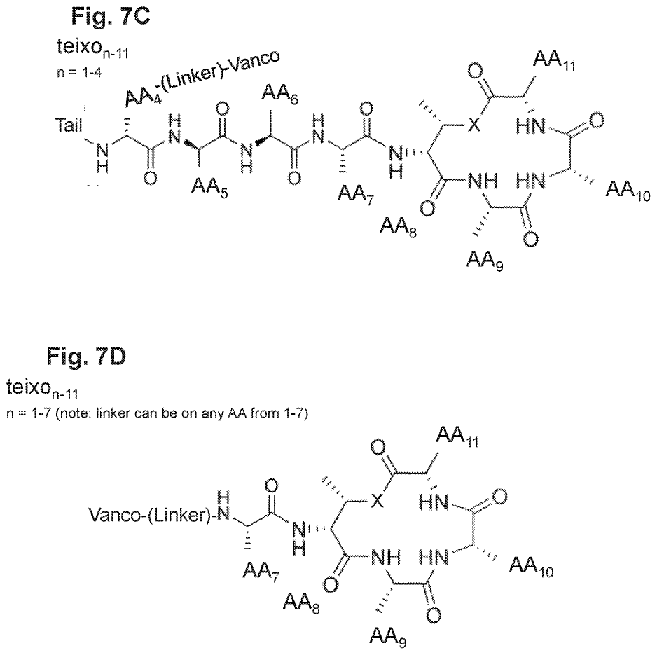

In some embodiments, a teixobactin ring comprises the natural occurring alanine (Ala) at residue position 9 (AA₉). In some embodiments, a teixobactin ring comprises a substituted amino acid at residue position 9. In various embodiments, AA₉ is an amino acid having a sidechain that comprises an amino group. In various embodiments, AA₉ is diamino propionic acid (Dap), 2,4-diamino butyric acid, ornithine (Orn), lysine (Lys), 6-azidolysine, or 2,7-diaminoheptanoic acid. In various embodiments, AA₉ is an amino acid having a sidechain that comprises a triazole group. In various embodiments, AA₉ is 1,2,3-triazole-4-carboxamide or 1,2,4-triazole-3-carboxamide. In various embodiments, AA₉ is an amino acid having a sidechain that comprises a carboxyl group. In various embodiments, AA₉ is aspartate (Asp) or glutamate (Glu). In various embodiments, AA₉ is an amino acid having a sidechain that comprises a thiol. In various embodiments, AA₉ is cysteine (Cys). In some embodiments, AA₉ is propargylglycine. In various embodiments, when AA₉ comprises an amino group, a triazole group, a carboxyl group, or a thiol group that is available, the available group is used to link to vancomycin to form the conjugate. In some embodiments, the vancomycin is linked to the teixobactin ring via AA₉ (FIG. 7A).

In some embodiments, a teixobactin ring comprises the naturally occurring allo-enduraciddine (allo-END) at residue position 10 (AA₁₀). In some embodiments, a teixobactin ring comprises a substituted amino acid at residue position 10. In various embodiments, AA₁₀ is an amino acid having a sidechain that comprises an amino group. In various embodiments, AA₁₀ is diamino propionic acid (Dap), 2,4-diamino butyric acid, ornithine (Orn), lysine (Lys), 6-azidolysine, or 2,7-diaminoheptanoic acid. In various embodiments, AA₁₀ is an amino acid having a sidechain that comprises a triazole group. In various embodiments, AA₁₀ is 1,2,3-triazole-4-carboxamide or 1,2,4-triazole-3-carboxamide. In various embodiments, AA₁₀ is an amino acid having a sidechain that comprises a carboxyl group. In various embodiments, AA₉ is aspartate (Asp) or glutamate (Glu). In various embodiments, AA₁₀ is an amino acid having a sidechain that comprises a thiol. In various embodiments, AA₁₀ is cysteine (Cys). In some embodiments, AA₁₀ is propargylglycine. In various embodiments, when AA₁₀ comprises an amino group, a triazole group, a carboxyl group, or a thiol group that is available, the available group is used to link to vancomycin to form the conjugate. In some embodiments, the vancomycin is linked to the teixobactin ring via AA₁₀ (FIG. 7B).

In some embodiments, a teixobactin ring comprises the naturally occurring isoleucine (Ile) at residue position 11 (AA₁₁). In some embodiments, a teixobactin ring comprises a substituted amino acid at residue position 11. In various embodiments, a teixobactin ring comprises valine (Val), leucine (Leu), or cyclohexylglycine (Chg) at residue position 11. In some embodiments, AA₁₁ is a bulky hydrophobic amino acid, which is any amino acid with a side chain that promotes hydrophobic interactions.

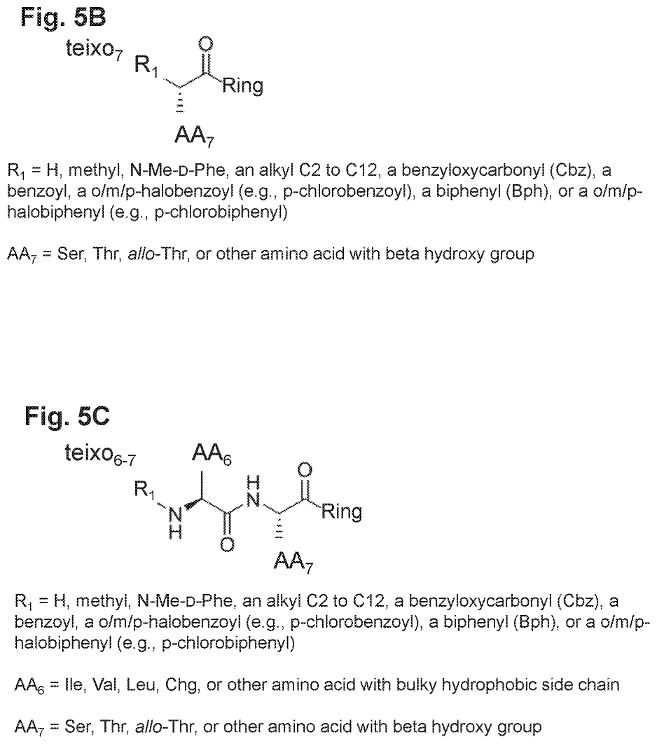

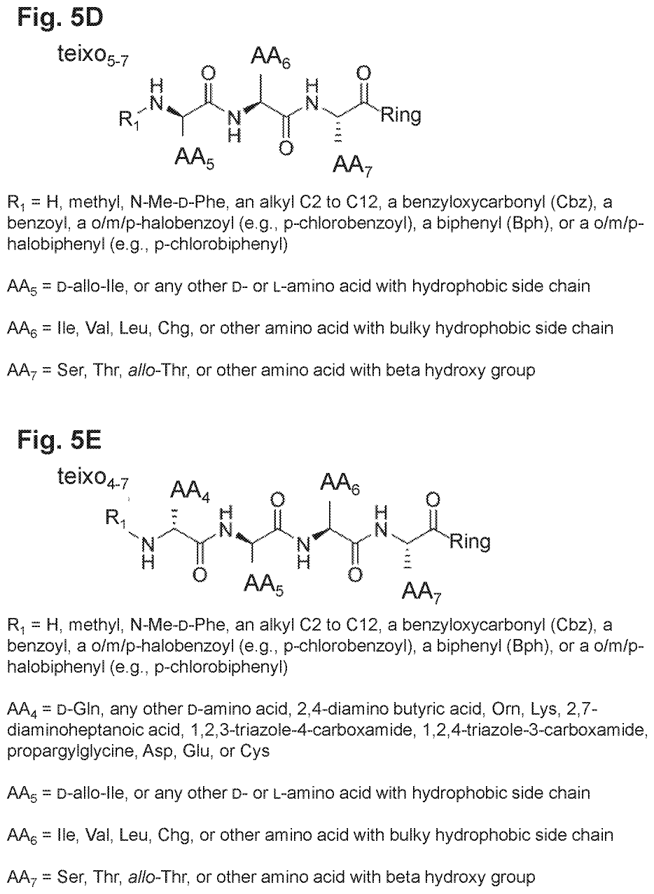

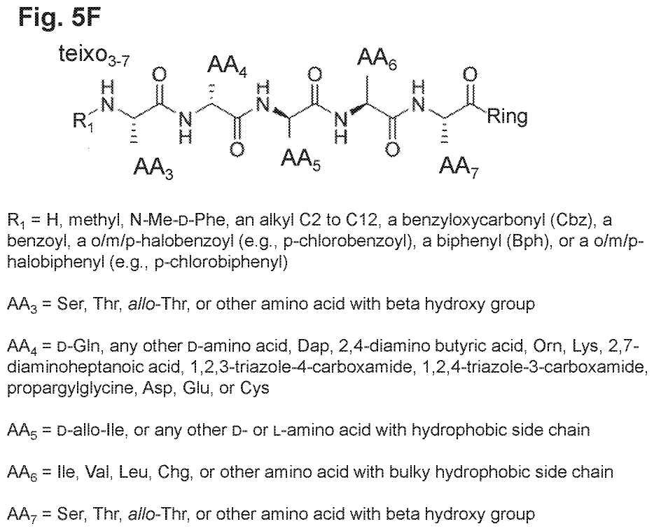

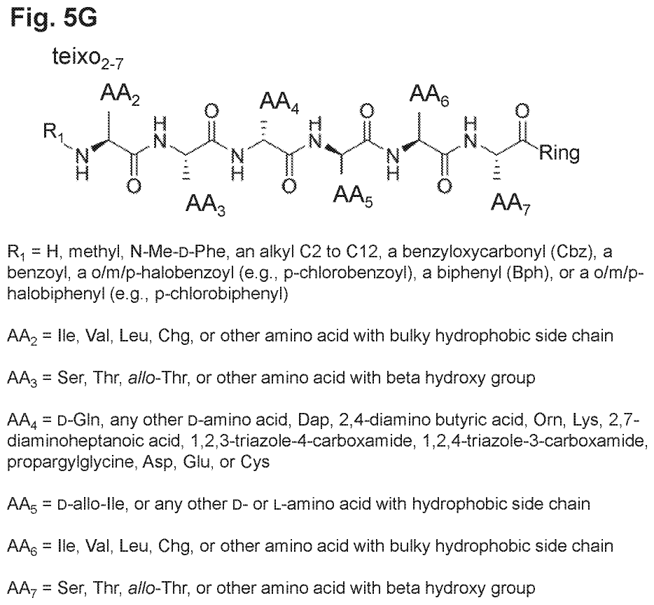

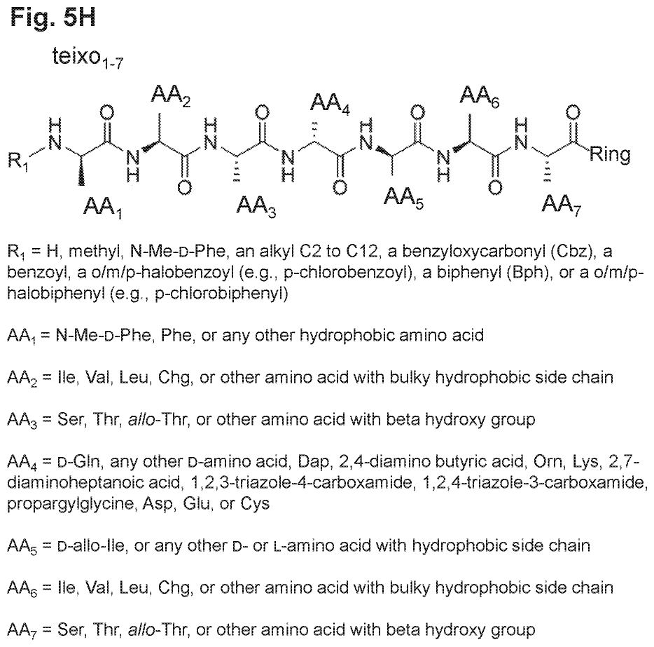

In several embodiments, a tail is linked to the N-terminus of the teixobactin ring (FIG. 4). In many embodiments, the tail comprises one or more amino acids. In some embodiments, the tail comprises a chain of amino acids from teixobactin. In some embodiments, the tail comprises the naturally occurring amino acids of teixobactin residues 1-7 (teixo₁₋₇) (FIG. 5A; SEQ ID NO: 4). In various embodiments, the tail comprises a sequence of amino acids derived from teixobactin or an analogue or teixobactin (teixoₓ₋₇), in which the N-terminus of the sequence begins with an amino acid at residue position 1-7 (i.e., x=1-7). In various embodiments, the tail comprises one of the following: teixo₇ (FIG. 5B), teixo₆₋₇ (FIG. 5C), teixo₅₋₇ (FIG. 5D), teixo₄₋₇ (FIG. 5E), teixo₃₋₇ (FIG. 5F), teixo₂₋₇ (FIG. 5G), or teixo₁₋₇ (FIG. 5H). The amino acid at each residue position can be the naturally occurring amino acid or an alternative substituent. The N-terminal amino acid of the teixobactin tail can optionally include a chemical moiety linked to its N-terminus, if available for linkage (e.g. if residue position 1 has the naturally occurring N-Me-d-Phe, the N-terminal amino acid does not have availability for further N-linkage).

In several embodiments, the tail is linked to the teixobactin ring (teixo₈₋₁₁; FIG. 3B) via the C-terminus of the amino acid at residue position 7 (i.e., AA₇), yielding a teixobactin comprising residue positions X-11 (teixoₓ₋₁₁) in which the N-terminus of the sequence begins with an amino acid at residue position 1-7 (i.e., x=1-7). In various embodiments, the tail plus the teixobactin ring comprises one of the following: teixo₇₋₁₁ (SEQ ID NO: 5), teixo₆₋₁₁ (SEQ ID NO: 6), teixo₅₋₁₁ (SEQ ID NO: 7), teixo₄₋₁₁ (SEQ ID NO: 8), teixo₃₋₁₁ (SEQ ID NO: 9), teixo₂₋₁₁ (SEQ ID NO: 10), or teixo₁₋₁₁ (SEQ ID NO: 11).

In some embodiments, a teixobactin tail comprises the naturally occurring N-Me-D-Phe at residue position 1 (AA₁). In some embodiments, a teixobactin ring structure comprises a substituted amino acid at residue position 1. In some embodiments, a teixobactin tail comprises phenyalanine (Phe) at residue position 1. In some embodiments, AA₁ is any other hydrophobic D-amino acid with an NH₂, N-methyl or N-alkyl substituent.

In some embodiments, a teixobactin tail comprises the naturally occurring isoleucine (Ile) at residue position 2 (AA₂). In some embodiments, a teixobactin ring structure comprises a substituted amino acid at residue position 2. In various embodiments, a teixobactin tail comprises valine (Val), leucine (Leu), or cyclohexylglycine (Chg) at residue position 2. In some embodiments, AA₂ is a bulky hydrophobic amino acid, which is any amino acid with a side chain that promotes hydrophobic interactions.

In some embodiments, a teixobactin tail comprises the naturally occurring serine (Ser) at residue position 3 (AA₃). In some embodiments, a teixobactin tail comprises a substituted amino acid at residue position 3. In various embodiments, a teixobactin tail comprises threonine (Thr) or allo-threonine (allo-Thr) at residue position 3. In some embodiments, AA₃ is any other amino acid having a beta hydroxy group.

In some embodiments, a teixobactin tail comprises the naturally occurring D-glutamine (D-Gln) at residue position 4 (AA₄). In some embodiments, a teixobactin tail comprises a substituted amino acid at residue position 4. In various embodiments, AA₄ is an amino acid having a sidechain that comprises an amino group. In various embodiments, AA₄ is diamino propionic acid (Dap), 2,4-diamino butyric acid, ornithine (Orn), lysine (Lys), 6-azidolysine, or 2,7-diaminoheptanoic acid. In various embodiments, AA₄ is an amino acid having a sidechain that comprises a triazole group. In various embodiments, AA₄ is 1,2,3-triazole-4-carboxamide or 1,2,4-triazole-3-carboxamide. In various embodiments, AA₄ is an amino acid having a sidechain that comprises a carboxyl group. In various embodiments, AA₄ is aspartate (Asp) or glutamate (Glu). In various embodiments, AA₄ is an amino acid having a sidechain that comprises a thiol. In some embodiments, AA₄ is cysteine (Cys). In some embodiments, AA₄ is propargylglycine. In various embodiments, when AA₄ comprises an amino group, a triazole group, a carboxyl group, or a thiol group that is available, the available group is used to link to vancomycin to form the conjugate. In some embodiments, the vancomycin is linked to the teixobactin ring via AA₄ (FIG. 7C).

In some embodiments, a teixobactin tail comprises the naturally occur D-allo-isoleucine (D-allo-Ile) at residue position 5 (AA₅). In some embodiments, a teixobactin tail comprises a substituted amino acid at residue position 5. In various embodiments, AA₅ is a D-amino acid with a hydrophobic side chain.

In some embodiments, a teixobactin tail comprises the naturally occurring isoleucine (Ile) at residue position 6 (AA₆). In some embodiments, a teixobactin ring structure comprises a substituted amino acid at residue position 6. In various embodiments, a teixobactin tail comprises valine (Val), leucine (Leu), or cyclohexylglycine (Chg) at residue position 6. In some embodiments, AA₆ is a bulky hydrophobic amino acid, which is any amino acid with a side chain that promotes hydrophobic interactions.

In some embodiments, a teixobactin tail comprises the naturally occurring serine (Ser) at residue position 7 (AA₇). In some embodiments, a teixobactin tail comprises a substituted amino acid at residue position 7. In various embodiments, a teixobactin tail comprises threonine (Thr) or allo-threonine (allo-Thr) at residue position 7. In some embodiments, AA₇ is any other amino acid having a beta hydroxy group.

Various embodiments of vancomycin-teixobactin conjugates can comprise a teixobactin sequence that is modified with one more substituted amino acids. A substitution is a change of amino acid, a structural alteration of amino acid, an insertion of an amino acid, or a deletion of an amino acid. In some embodiments, a teixobactin sequence comprises one or more substitutions. In some embodiments, a teixobactin sequence has no more than one substitution. In some embodiments, a teixobactin sequence comprises two or more substitutions. In some embodiments, a teixobactin sequence has no more than two substitutions. In some embodiments, a teixobactin sequence comprises three or more substitutions. In some embodiments, a teixobactin sequence has no more than three substitutions. In some embodiments, a teixobactin sequence comprises four or more substitutions. In some embodiments, a teixobactin sequence has no more than four substitutions. In some embodiments, a teixobactin sequence comprises five or more substitutions. In some embodiments, a teixobactin sequence has no more than five substitutions. In some embodiments, a teixobactin sequence comprises six or more substitutions. In some embodiments, a teixobactin sequence has no more than six substitutions. In some embodiments, a teixobactin sequence comprises seven or more substitutions. In some embodiments, a teixobactin sequence has no more than seven substitutions. In some embodiments, a teixobactin sequence comprises eight or more substitutions. In some embodiments, a teixobactin sequence has no more than eight substitutions. In some embodiments, a teixobactin sequence comprises nine or more substitutions. In some embodiments, a teixobactin sequence has no more than nine substitutions. In some embodiments, a teixobactin sequence comprises ten or more substitutions. In some embodiments, a teixobactin sequence has no more than ten substitutions. In some embodiments, a teixobactin sequence comprises eleven substitutions. Any substitution described herein can be combined with any other substitution(s) described herein that still yield a teixobactin analogue.

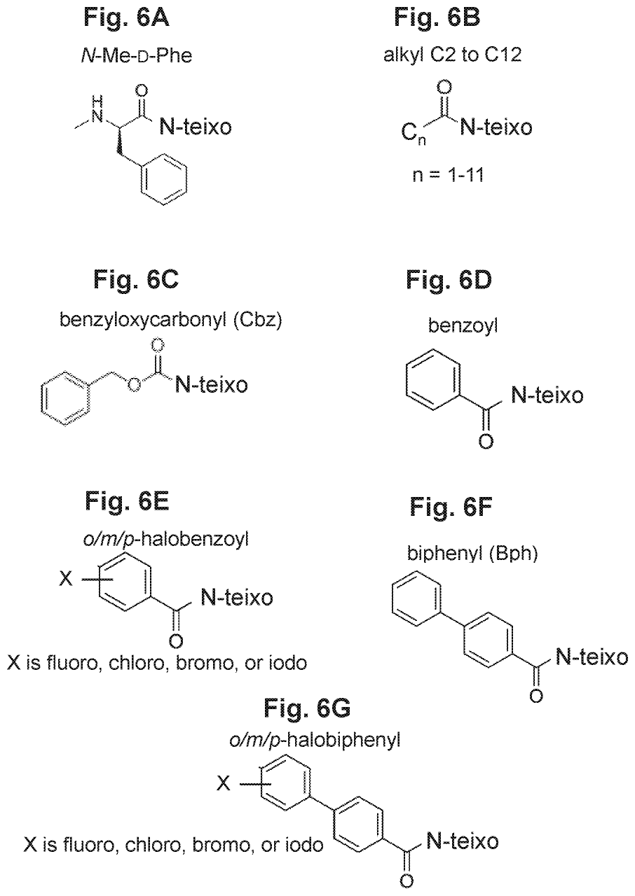

In some embodiments, a teixobactin tail comprises moiety at the N-terminus. In various embodiments, the moiety at the N-terminus is hydrogen, a methyl group, N-Me-D-Phe (FIG. 6A), an alkyl C2 to C12 (FIG. 6B), a benzyloxycarbonyl (Cbz) (FIG. 6C), a benzoyl (FIG. 6D), a o/m/p-halobenzoyl (FIG. 6E), a biphenyl (Bph) (FIG. 6F), or a o/m/p-halobiphenyl (FIG. 6G).

Various embodiments are directed towards enantiomers of teixobactin ring structures and teixobactin tail structures. It has been found that enantiomer conformations of teixobactin have similar antibiotic activity to their standard counterpart (see, e.g., H. Yang, K. H. Chen, and J. S. Nowick, ACS Chem Biol. 2016; 11:1823-6, the disclosure of which is incorporated herein by reference).

Various embodiments are directed to further substitutions of teixobactin and teixobactin analogues. Many examples of substitutions of teixobactin and teixobactin analogues have been described in art (see, e.g., J. A. Karas, et al., Ann N Y Acad Sci. 2020; 1459:86-105; and H. Yang, et al., Chem Commun (Camb). 2017; 53:2772-2775; the disclosures of which are each incorporated herein by reference).

In many embodiments, a functional group of an amino acid (side chain, amino groups, or carboxyl groups), of a teixobactin is utilized to conjugate with vancomycin. Provided in FIGS. 7A to 7C are examples of teixobactin in which the side chain of an amino acid is used to conjugate with vancomycin. In some embodiments, the side chain of AA₉ is conjugated with vancomycin. In some embodiments, the side chain of AA₁₀ is conjugated with vancomycin. In some embodiments, the side chain of AA₄ is conjugated with vancomycin. Provided in 7D is an example of teixobactin in which the N-terminus I sued to conjugated with vancomycin. As noted previously, conjugation between the texiobactin ring structure and vancomycin can optionally include a linker between the two structures.

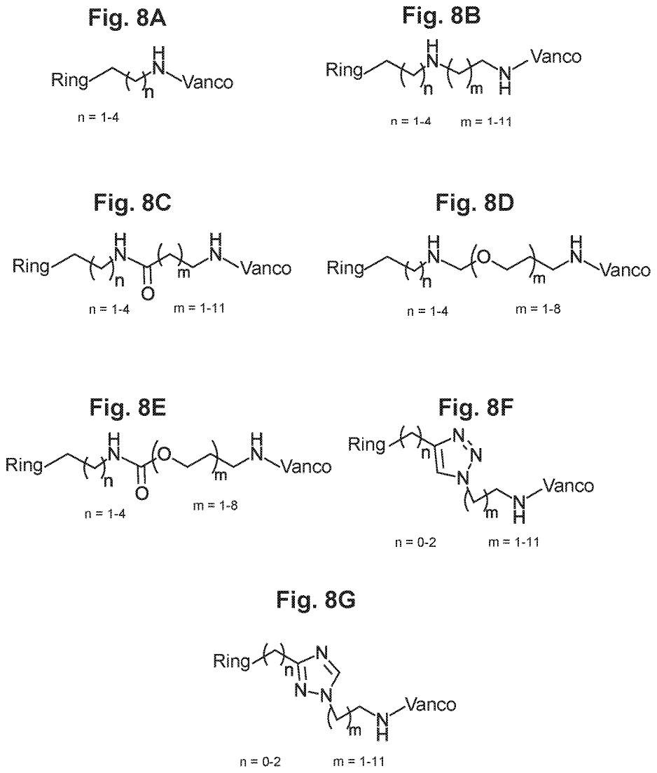

Various embodiments of vancomycin-teixobactin conjugates can comprise a linker between vancomycin and a teixobactin ring. Provided in FIGS. 8A to 8G are examples of structures that can be utilized to conjugate vancomycin and a teixobactin ring. These examples include a teixobactin amino acid side chain without a linker (FIG. 8A), a teixobactin amino acid side chain with a C2-C8 alkyl linker (FIG. 8B), a teixobactin amino acid side chain with a C2-C8 alkyl amino acid linker (FIG. 8C), a teixobactin amino acid side chain with a PEG linker (FIG. 8D), a teixobactin amino acid side chain with a PEG amino acid linker (FIG. 8E), a teixobactin 1,2,3-triazol amino acid with a linker (FIG. 8F), and a teixobactin 1,2,4-triazol amino acid with a linker (FIG. 8G). These examples utilize amino groups to form an amide bond with a carboxyl group of vancomycin, such as R³ of the structure within FIG. 8. As can be readily appreciated, carboxyl groups can be used to form an amide bond with an amino group of vancomycin, such as R¹, R⁴, or N-substituted R³.

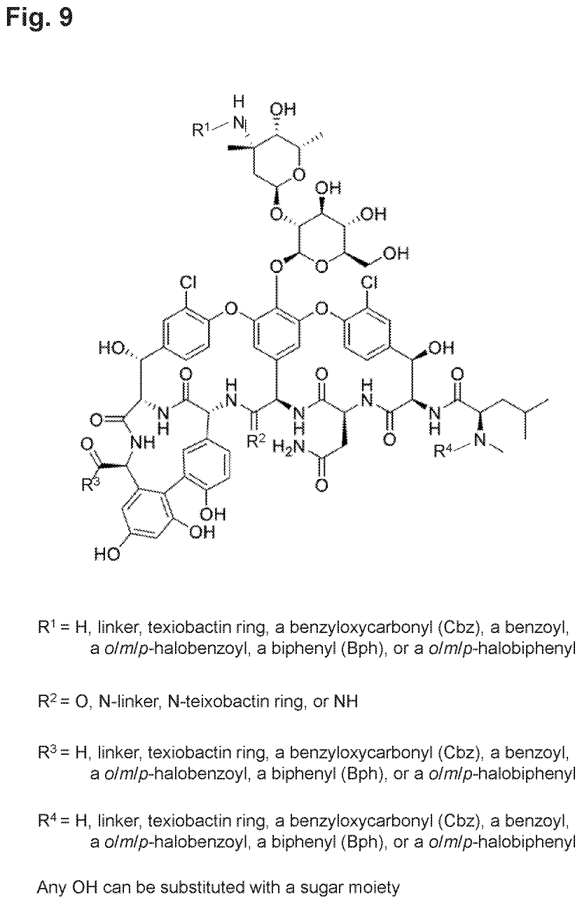

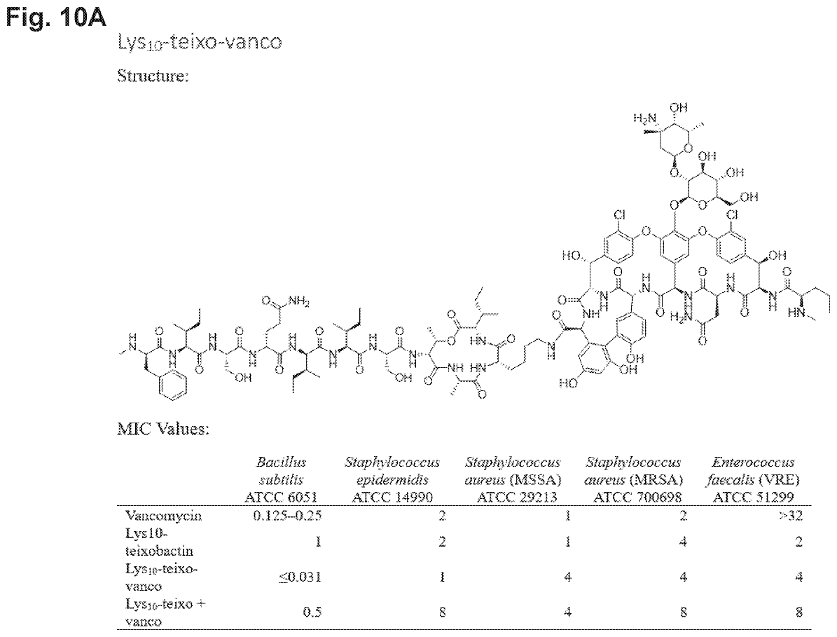

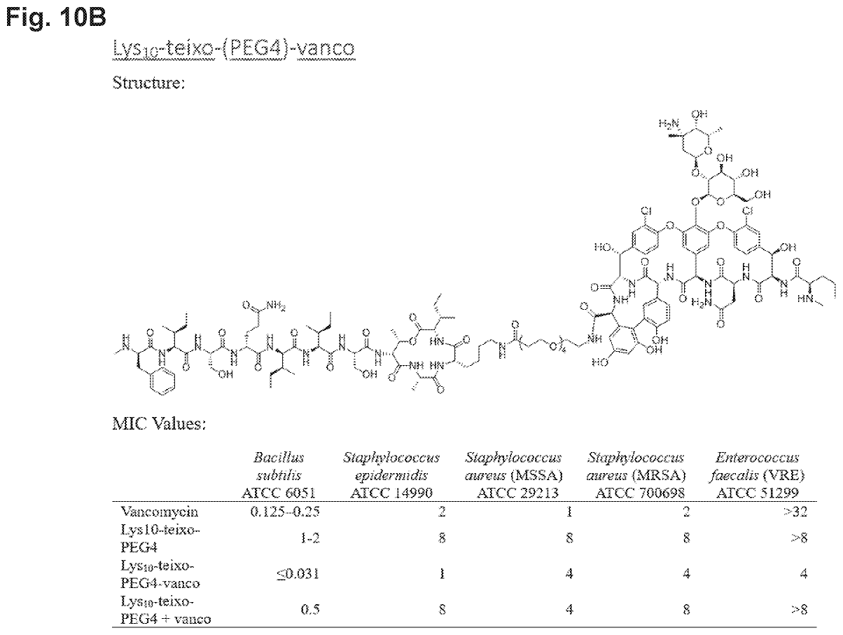

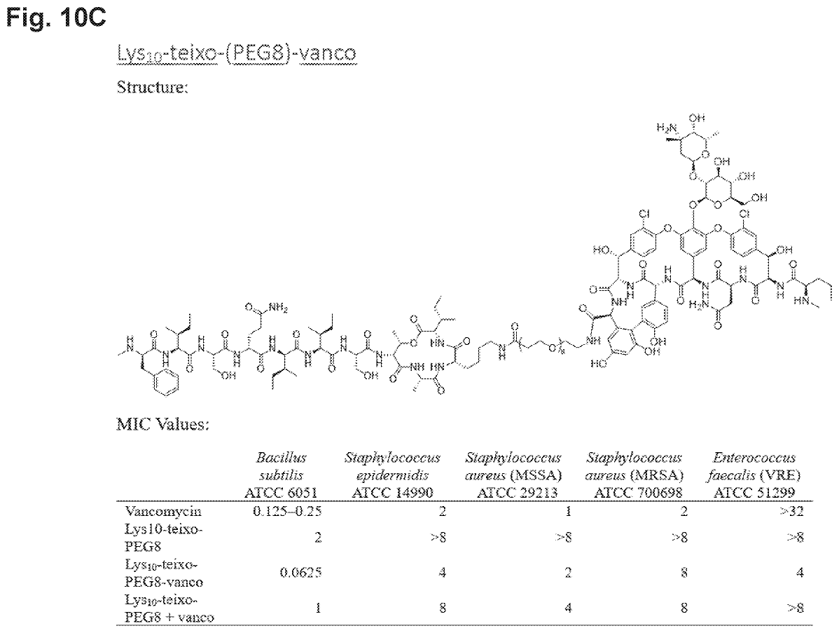

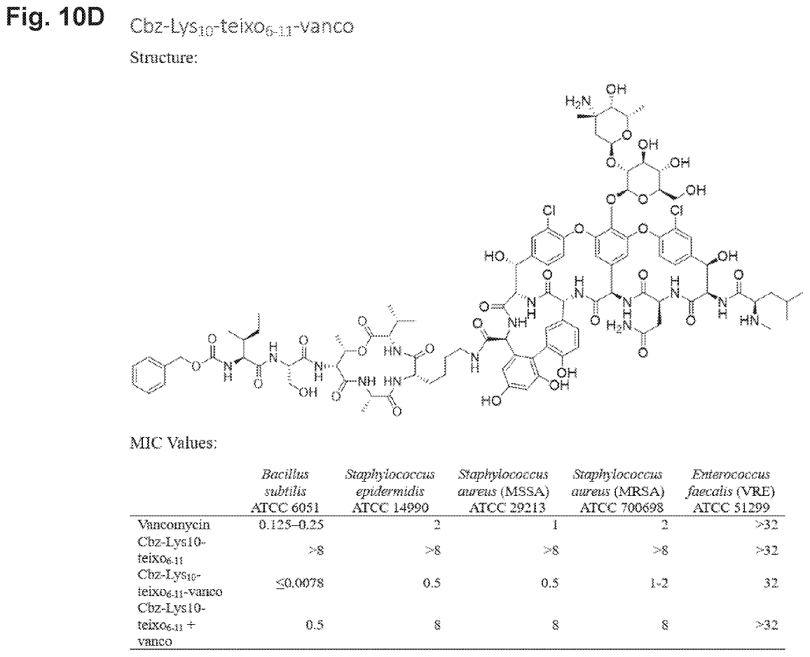

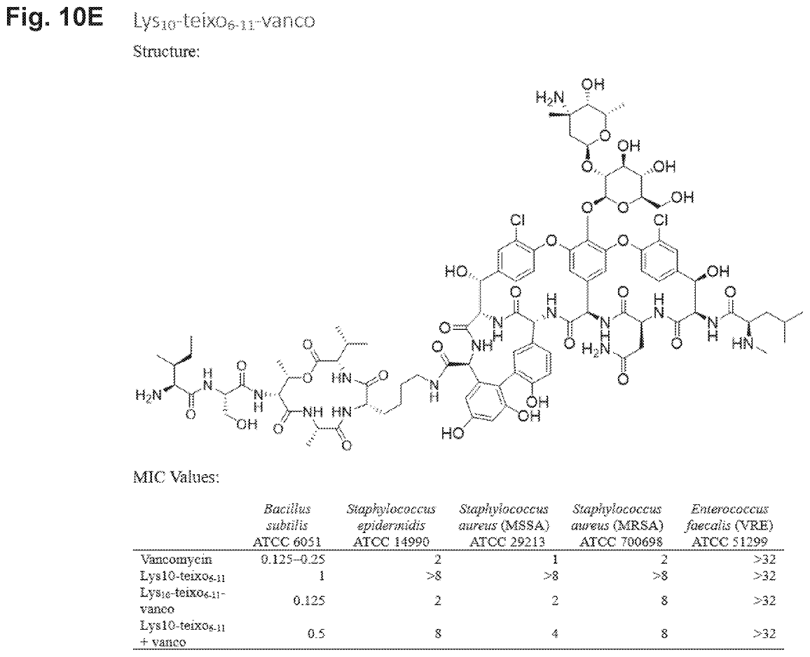

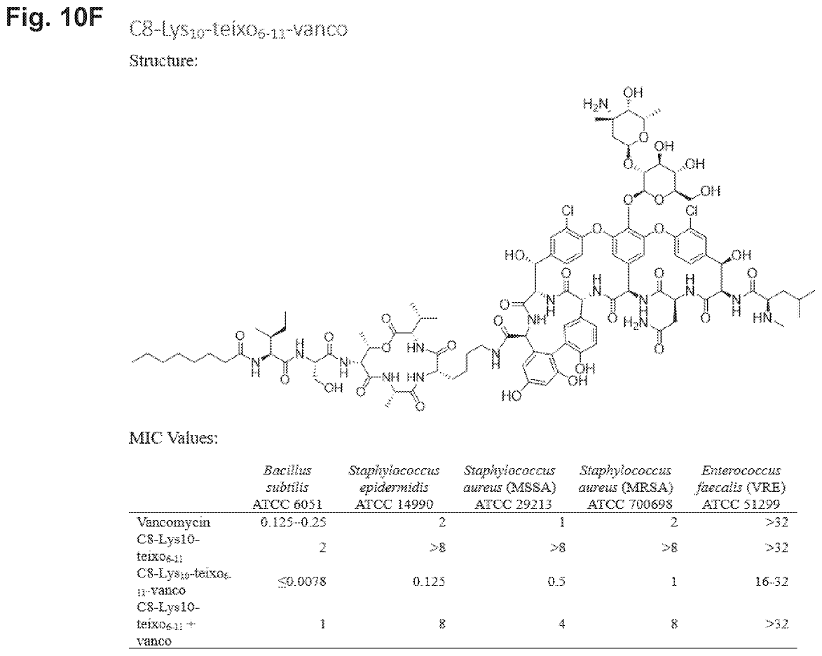

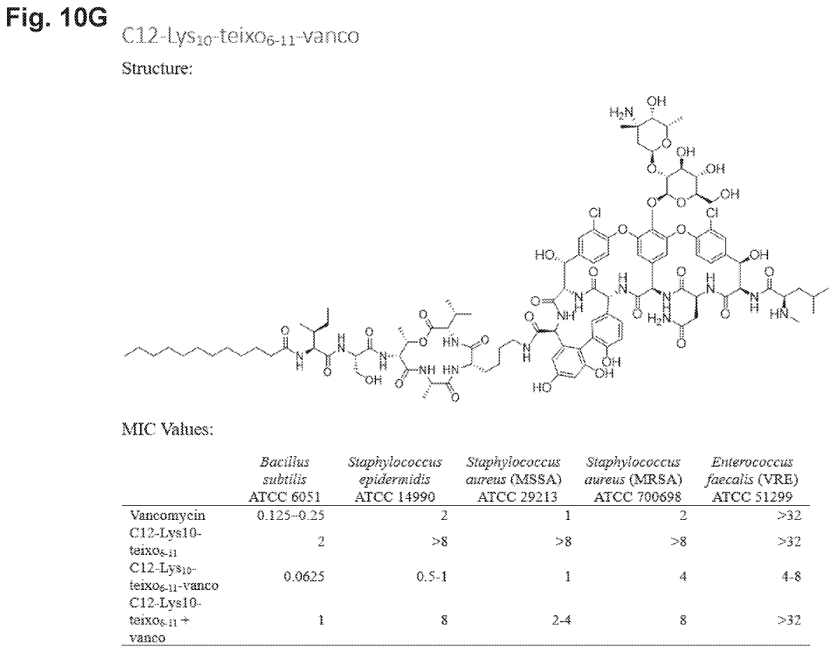

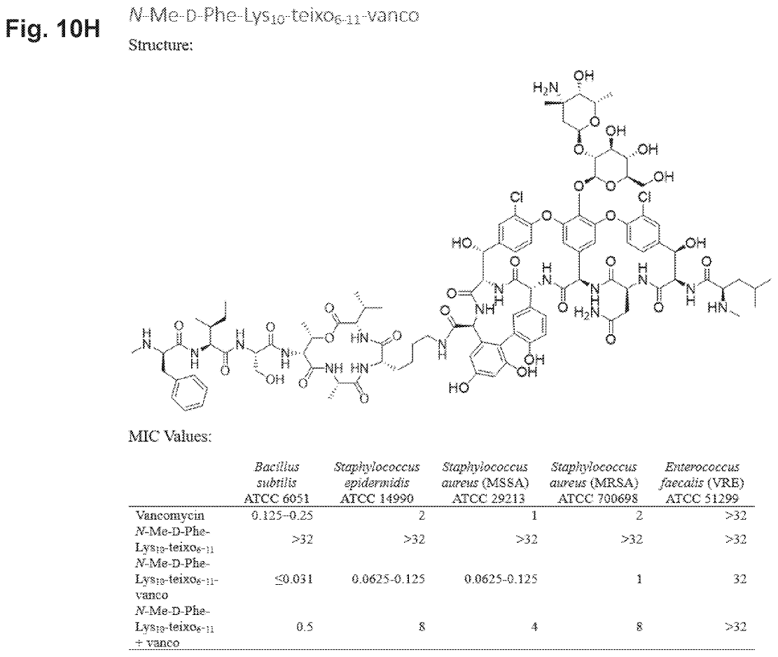

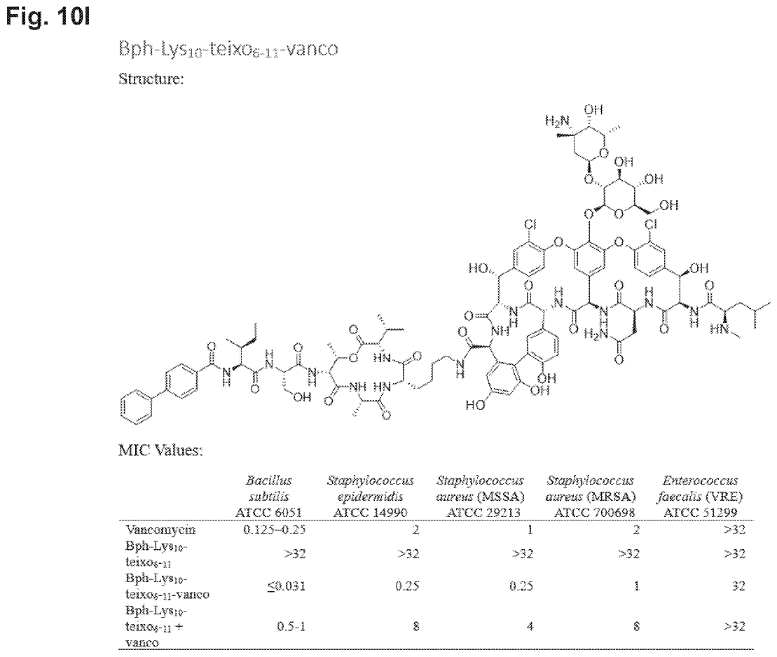

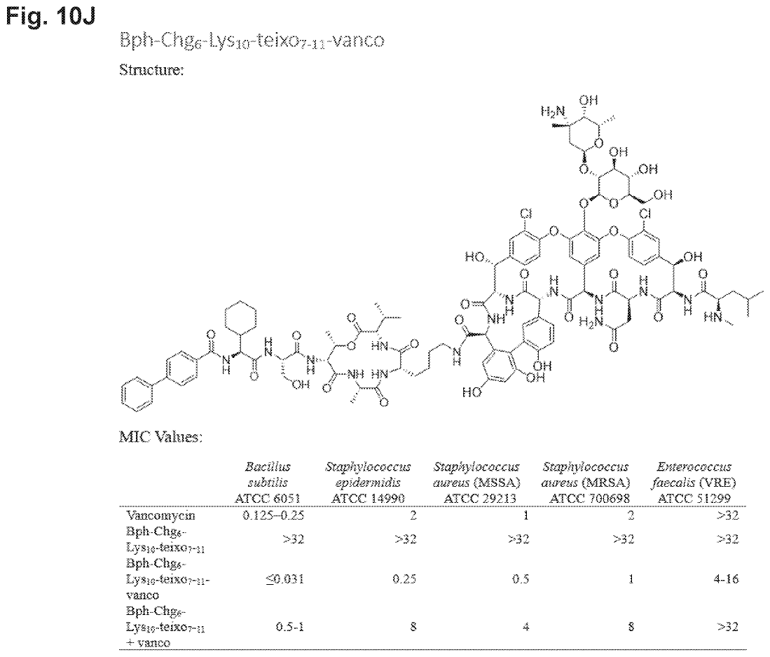

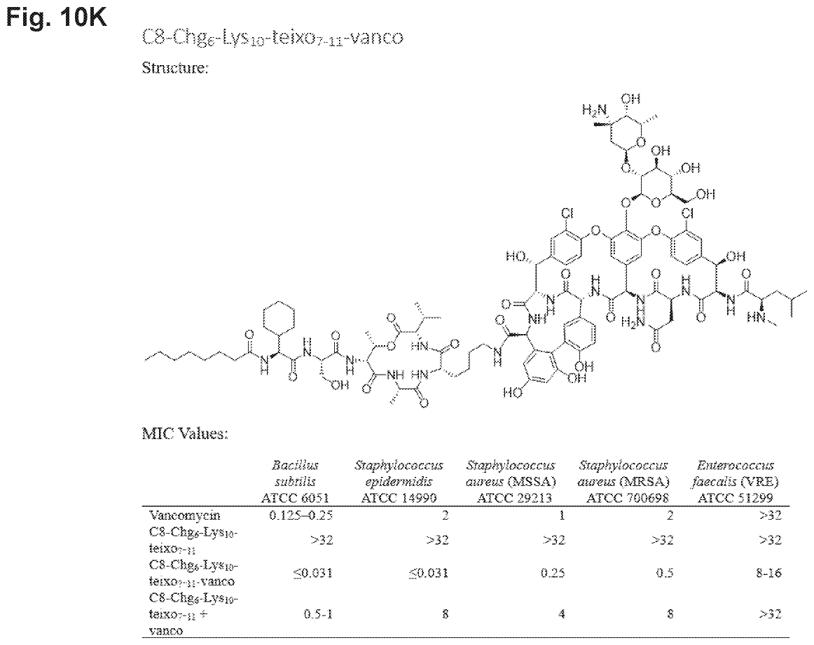

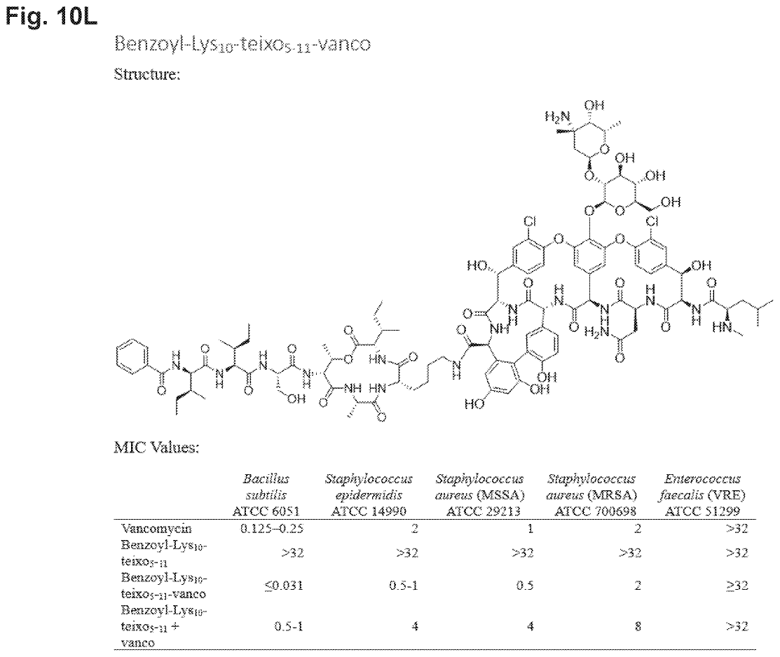

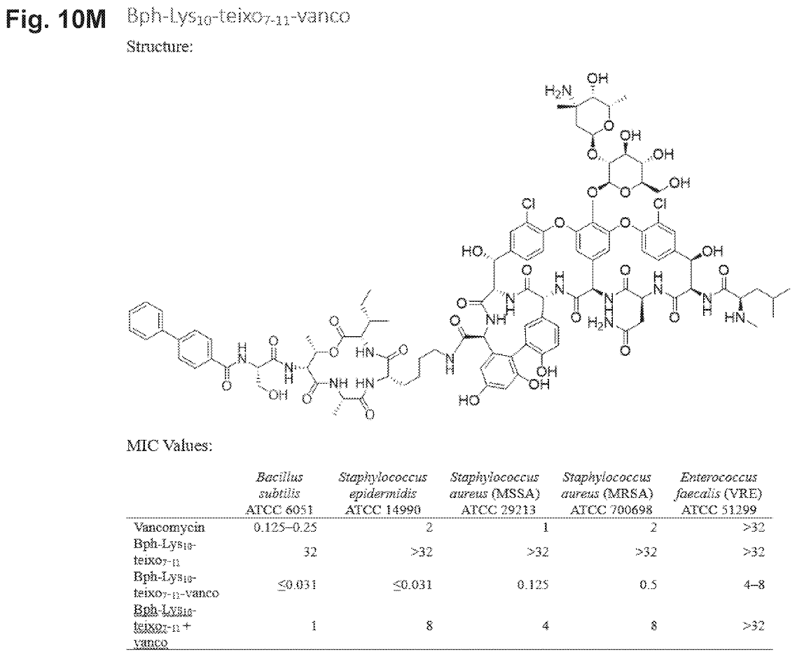

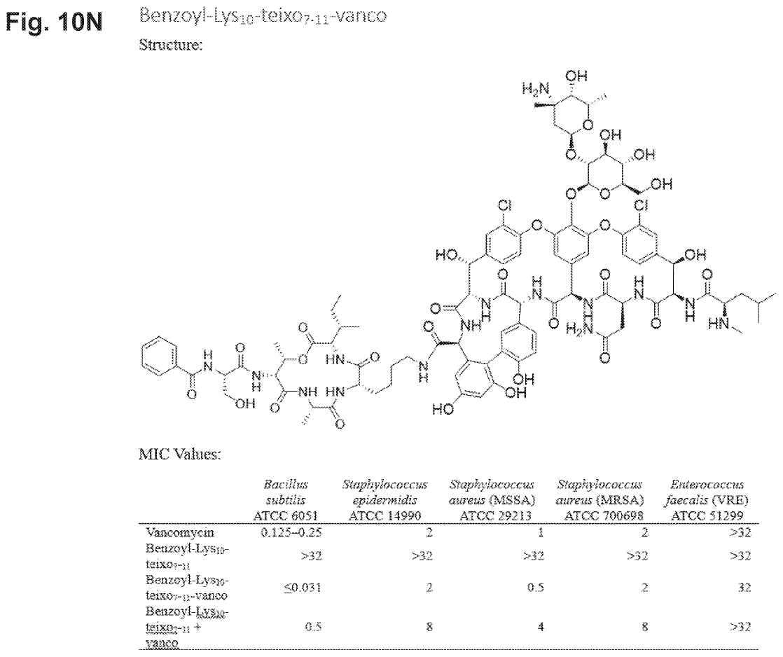

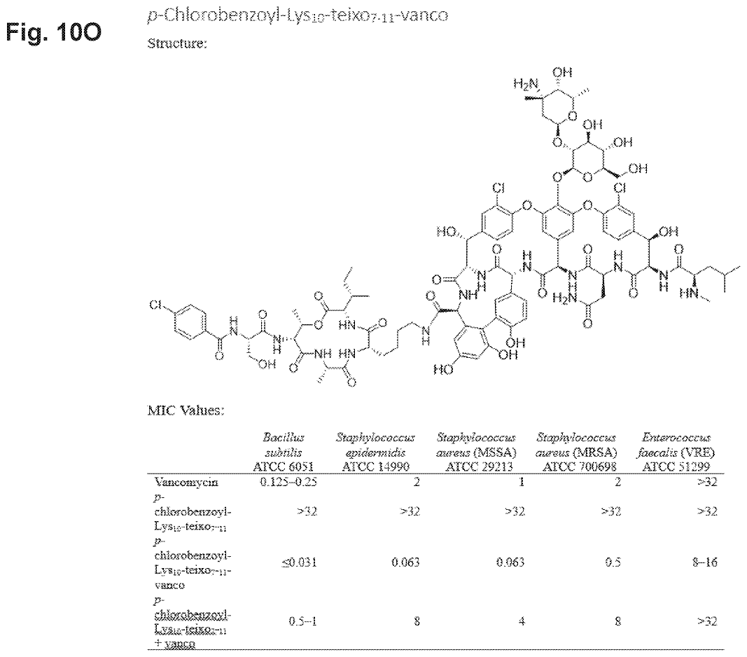

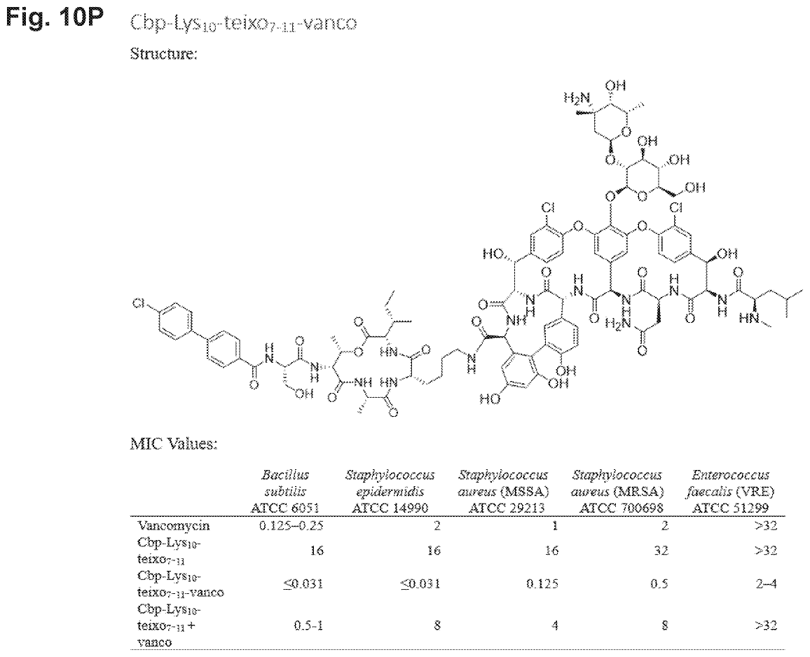

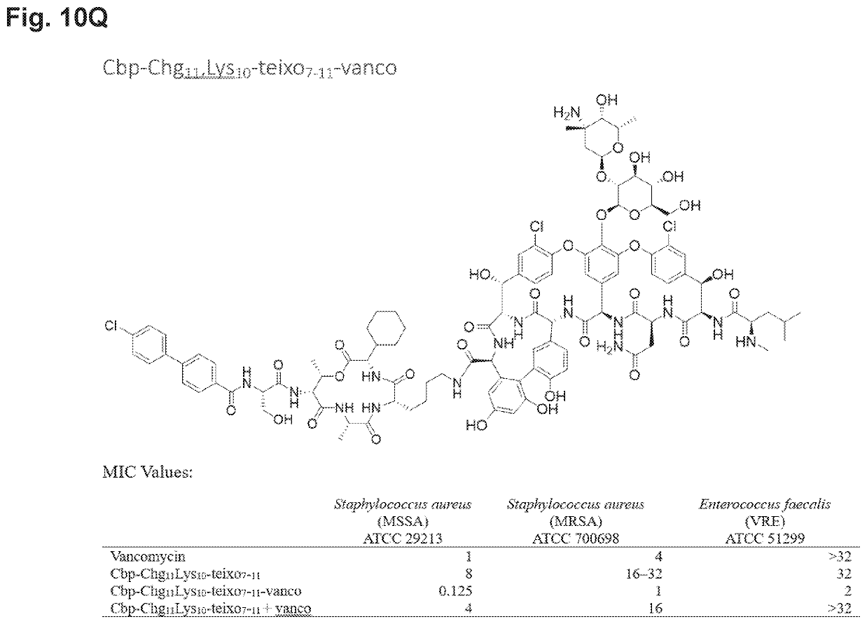

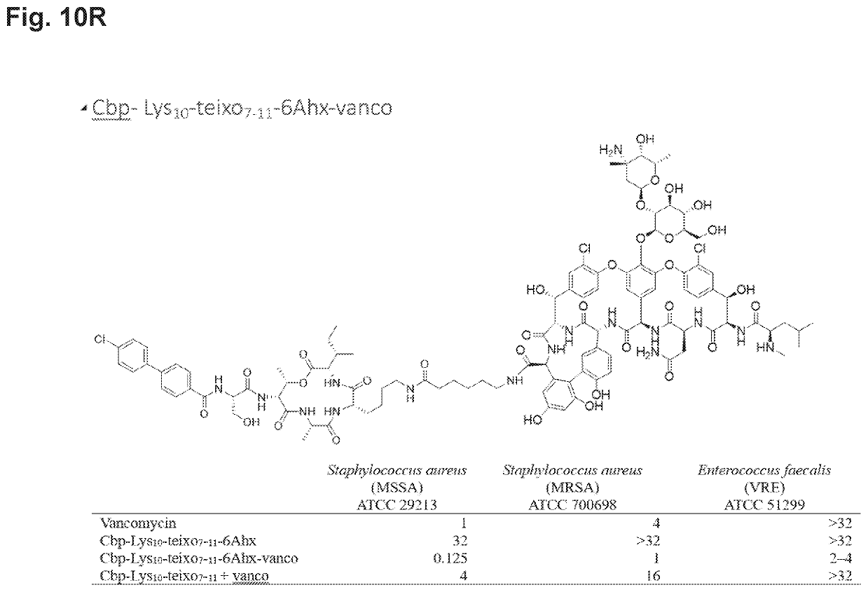

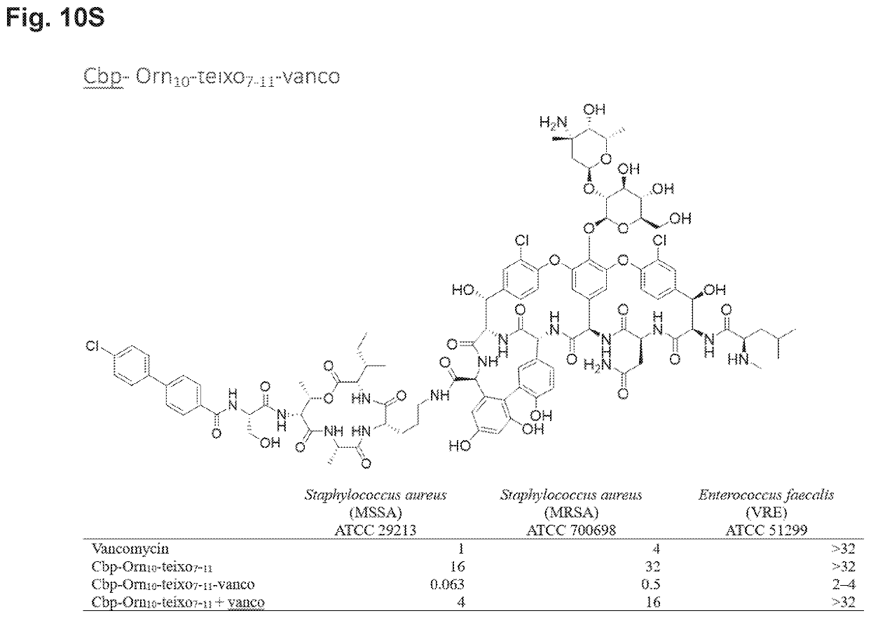

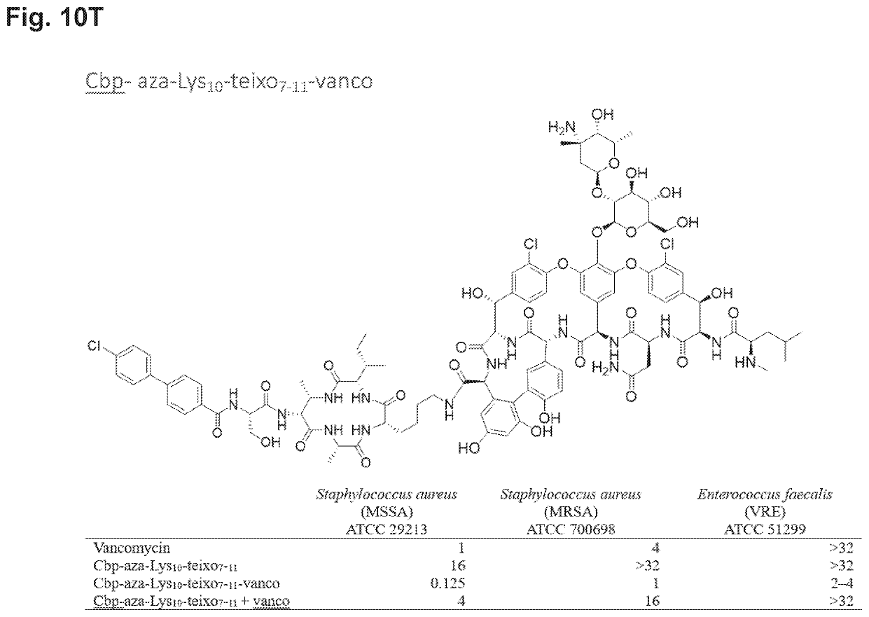

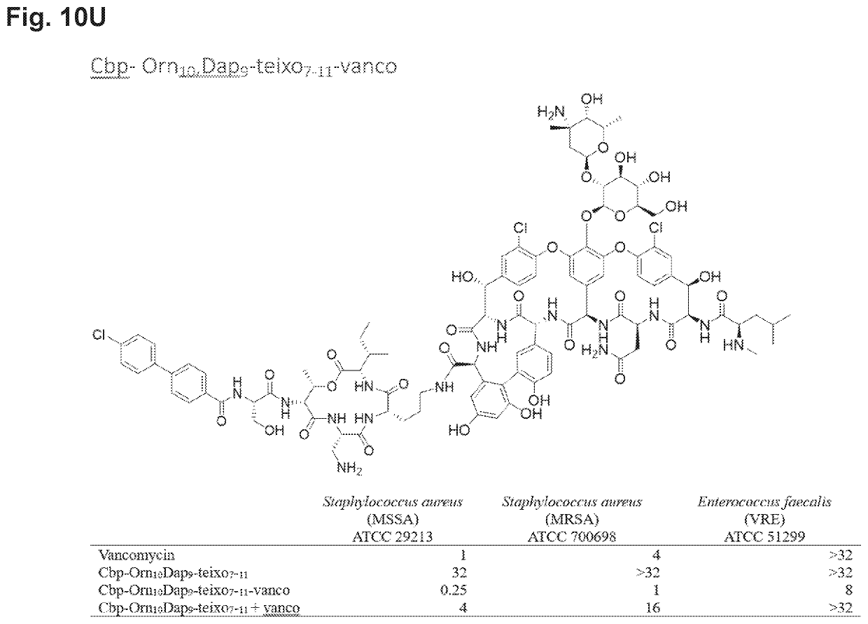

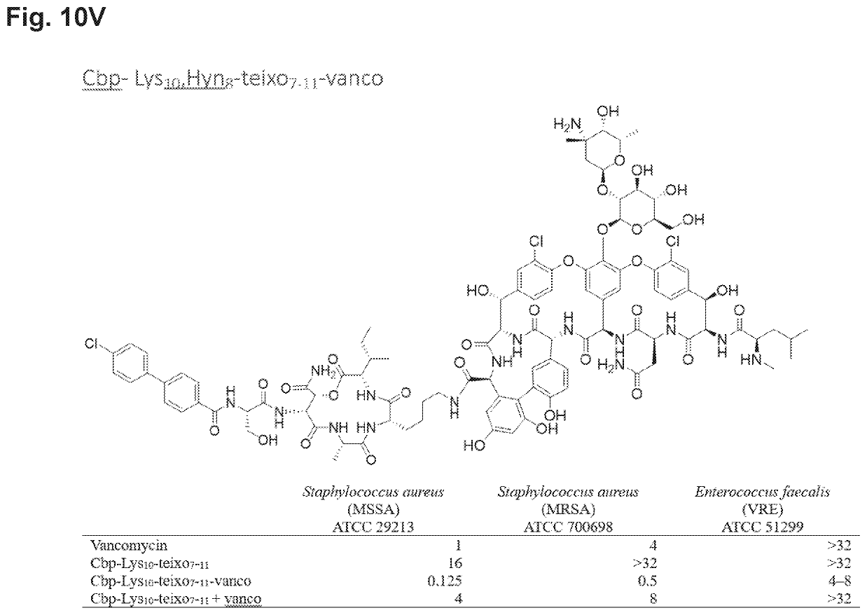

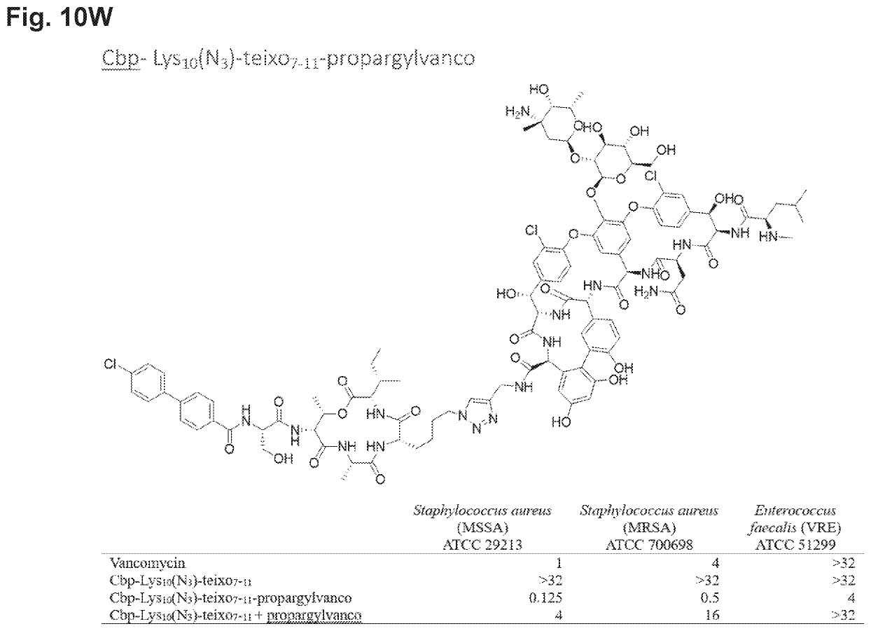

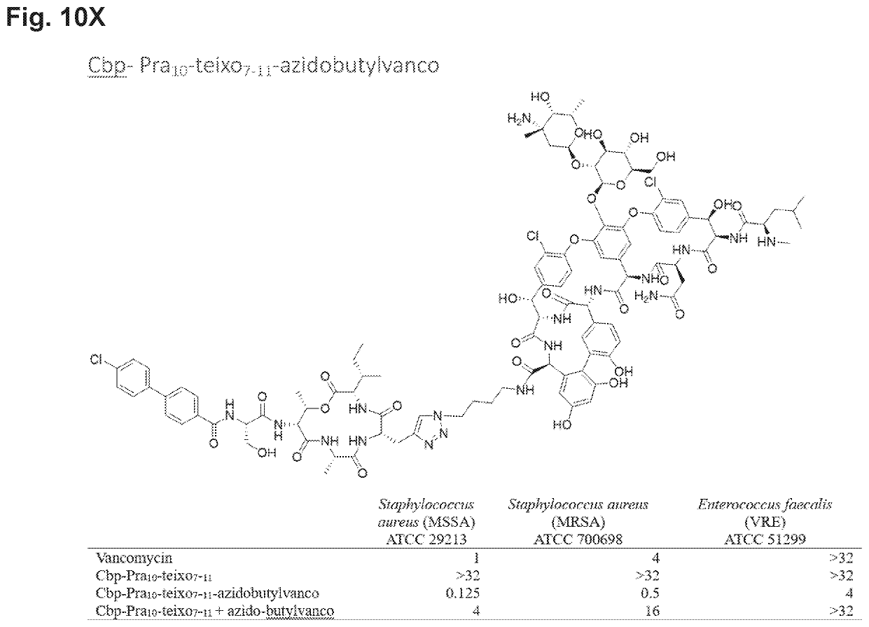

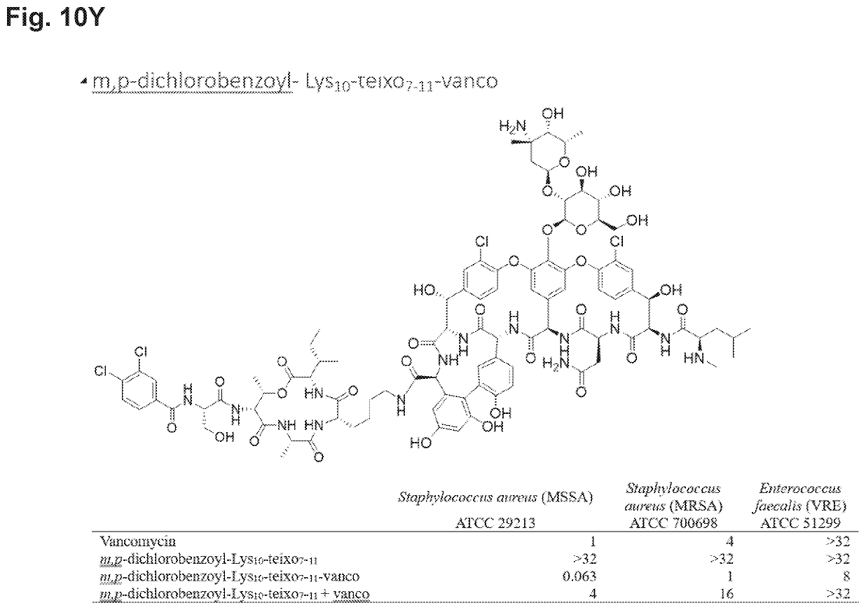

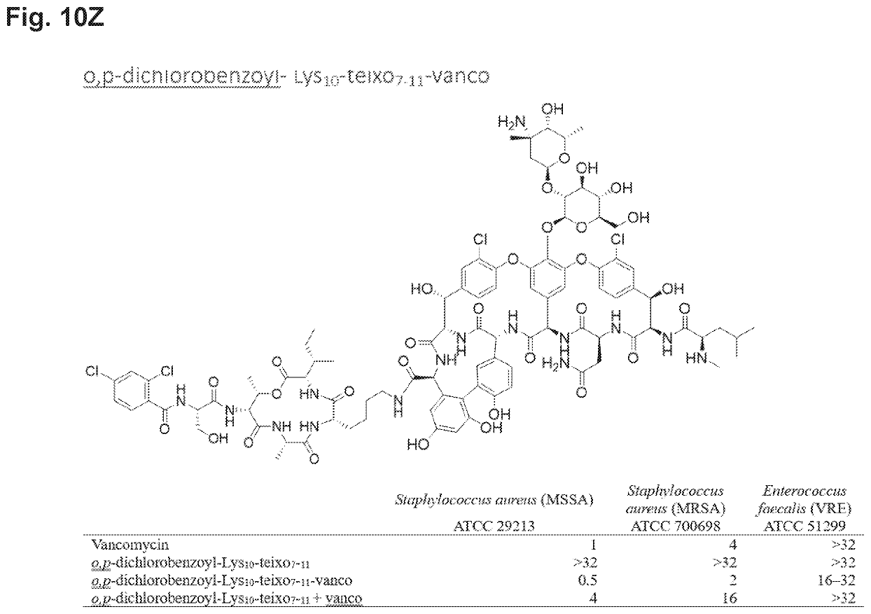

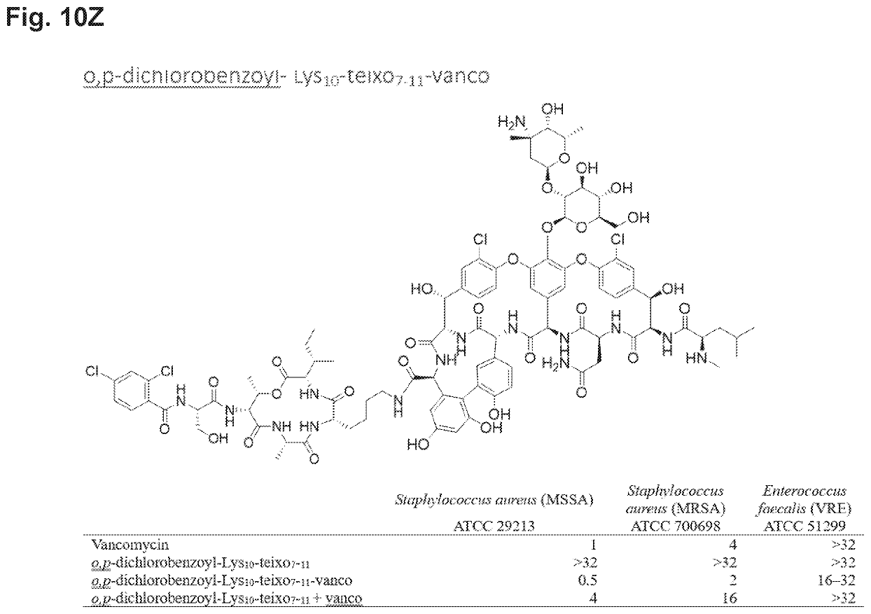

Several embodiments of vancomycin-teixobactin conjugates comprise a vancomycin conjugated to a teixobactin ring. Vancomycin can be a naturally occurring structure or an analogue structure. Provided in FIG. 9 is an example of various substitutions of various vancomycin analogues. Various locations of Vancomycin can be utilized to conjugate to a teixobactin ring, such as (for example) R¹, R², R³, or R⁴. Alternatively, when not utilized for linkage, R¹, R³, or R⁴ can independently be substituted with a benzyloxycarbonyl (Cbz), a benzoyl, a olm/p-halobenzoyl, a biphenyl (Bph), or a olm/p-halobiphenyl. R² can be substituted with N-linker or N-teixobactin ring when used for linkage or with NH when not used for linkage. Any hydroxyl group can be substituted with a sugar moiety.

Many analogues of vancomycin have been described within the literature and can be utilized to conjugate to a teixobactin (See, e.g., J. Chang, et al., ChemMedChem. 2013 June; 8 (6): 976-84; V. Yarlagadda, et al., J Med Chem. 2014 Jun. 12; 57 (11): 4558-68; A. Okano, et al., J Am Chem Soc. 2015 Mar. 18; 137 (10): 3693-704; S. Yoganathan and S. J. Miller, J Med Chem. 2015 Mar. 12; 58 (5): 2367-77; V. Yarlagadda, et al., ACS Infect Dis. 2016 Feb. 12; 2 (2): 132-9; A. Okano, et al., Proc Natl Acad Sci USA. 2017 Jun. 27; 114 (26): E5052-E5061; S. M. Silverman, et al., Chemistry. 2017 Jan. 1; 23 (1): 79-83; A. Okano, et. al., Chem Rev. 2017 Sep. 27; 117 (18): 11952-11993; D. Guan, et al., Angew Chem Int Ed Engl. 2019 May 13; 58 (20): 6678-6682; G. Dhanda, et al., J Med Chem. 2019 Apr. 11; 62 (7): 3184-3205; Z. C. Wu, et al., ACS Infect Dis. 2020 Aug. 14; 6 (8): 2169-2180; E. Muhlberg, et al., Can J Microbiol. 2020 January; 66 (1): 11-16; Z. C. Wu and D. L. Boger, Acc Chem Res. 2020 Nov. 17; 53 (11): 2587-2599; I. S. Shchelik and K. Gademann, ACS Med Chem Lett. 2021 Oct. 18; 12 (12): 1898-1904; Y. Acharya, et al., Chem Commun (Camb). 2022 Feb. 8; 58 (12): 1881-1897; L. Zhai, et al., Molecules. 2022 Nov. 9; 27 (22): 7685; P. Sarkar, et al., Chem Sci. 2023 Jan. 6; 14 (9): 2386-2398; the disclosures of which are each hereby incorporated by reference).

### Therapeutic Formulations & Modes of Treatment

Several embodiments are directed to the use of a medicament comprising a vancomycin-teixobactin conjugate compound for the treatment of a bacterial infection, especially an infection of gram-positive bacteria, and especially of antibiotic-resistant bacteria. As described herein, a vancomycin-teixobactin conjugate compound has synergistic efficacy against gram-positive bacteria generally and several vancomycin-teixobactin conjugates are effective against bacteria that are resistant to vancomycin (e.g., vancomycin-resistant Enterococci). In some embodiments, a medicament comprising a vancomycin-teixobactin conjugate compound administered in a therapeutically effective amount to an individual as part of a course of treatment. Individuals are to include any animal, including (but not limited to) an animal, a mammal, a bird, a reptile, a primate, a human, a domesticated animal, a farm animal, or a zoo animal. As used in this context, to “treat” means to ameliorate at least one symptom of the disorder to be treated or to provide a beneficial physiological effect and is to include prophylactic treatments for prevention or mitigation of bacterial infection. For compounds for the treatment of bacterial infection, amelioration of a symptom could be inhibition of bacteria replication or reduction of one or more symptoms related to the infection of the pathogen. Assessment of amelioration can be performed in many ways, including, but not limited to assessing active bacterial infection (e.g., plate streaking), or reduction in pathogen related symptoms.

Numerous embodiments are directed to the use of a medicament comprising a vancomycin-teixobactin conjugate compound as an antibiotic. In several embodiments, an individual to be treated is infected with bacteria or is at risk of bacterial infection (e.g., prophylactic treatment). A number of bacterial pathogens can be treated. In some embodiments, a medicament comprising a vancomycin-teixobactin conjugate compound is administered to treat an individual with an infection of or at risk of being infected by a gram-positive pathogen, including (but not limited to) infections of Staphylococcus, Streptococcus, Clostridium, Bacillus, Corynebacterium, Enterococci, and Listeria. Various pathogenic Staphylococcus that can be treated include (but are not limited to) S. aureus and S. epidermidis. Various pathogenic Streptococcus that can be treated include (but are not limited to) S. pneumoniae, S. pyogenes and S. agalactiae. Various pathogenic Clostridium that can be treated include (but are not limited to) C. tetani, C. botulinum, C. perfringens and C. difficile. Various pathogenic Bacillus that can be treated include (but are not limited to) B. anthracis and B. cereus. Various pathogenic Corynebacterium that can be treated include (but are not limited to) C. diphtheria. Various pathogenic Listeria that can be treated include (but are not limited to) L. monocytogenes. Furthermore, in accordance with some embodiments, a medicament comprising a vancomycin-teixobactin conjugate compound is administered to treat an individual with an infection of difficult-to-treat pathogens such as (for example) Bacillus anthracis (Anthrax), Staphylococcus aureus (MRSA), vancomycin-resistant Enterococci (VRE), and multidrug-resistant Mycobacterium tuberculosis (MDR-TB). In some embodiments, a medicament comprising a vancomycin-teixobactin conjugate compound is for the specific treatment vancomycin-resistant bacteria, such as vancomycin-resistant Enterococci (VRE).

A therapeutically effective amount can be an amount sufficient to prevent reduce, ameliorate or eliminate the symptoms of diseases or pathological conditions susceptible to such treatment. In some embodiments, a therapeutically effective amount is an amount sufficient to inhibit bacterial replication in an infected individual or mitigate infection in a prophylactic treatment.

Dosage, toxicity and therapeutic efficacy of the compounds can be determined, e.g., by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred. While compounds that exhibit toxic side effects may be used, care should be taken to design a delivery system that targets such compounds to the site of affected tissue in order to minimize potential damage to non-infected cells. In many embodiments, therapeutic formulations comprising a vancomycin-teixobactin conjugate compound are administered at a greater dosage concentration than therapeutic formulations comprising unconjugated teixobactin in which dosage concentration may be limited due to aggregation of teixobactin. In some embodiments, therapeutic formulations comprising a vancomycin-teixobactin conjugate compound are administered at a lesser dosage concentration than therapeutic formulations comprising unconjugated vancomycin, unconjugated teixobactin, or mixtures of unconjugated vancomycin and unconjugated teixobactin in which higher dosage concentrations are necessary to achieve the same effectiveness that is achieved by the synergistic effect of conjugating teixobactin with vancomycin.

Data obtained from cell culture assays or animal studies can be used in formulating a range of dosage for use in a subject. If the medicament is provided systemically, the dosage of such compounds lies preferably within a range of circulating concentrations that include the ED₅₀ with little or no toxicity. The dosage may vary within this range depending upon the dosage form employed and the route of administration utilized. For any compound used in a method of the various embodiments, the therapeutically effective dose can be estimated initially from cell culture assays. A dose may be formulated in animal models to achieve a circulating plasma concentration or within the local environment to be treated in a range that includes the IC₅₀ (i.e., the concentration of the test compound that achieves a half-maximal inhibition of bacterial replication) or MIC (i.e., the lowest concentration of the test compound that stops visible growth) as determined in cell culture. Such information can be used to more accurately determine useful doses in humans. Levels in plasma may be measured, for example, by liquid chromatography coupled to mass spectrometry. In some embodiments, a therapeutic effect is achieved with an IC₅₀ less than 2.0 μg/mL, less than 1.5 g/mL, less than 1.0 μg/mL, less than 0.5 μg/mL, less than 0.25 μg/mL, or less than 0.125 μg/mL. In some embodiments, a therapeutic effect is achieved with a MIC less than 4.0 μg/mL, less than 2.0 μg/mL, less than 1.5 μg/mL, less than 1.0 μg/mL, less than 0.5 μg/mL, less than 0.25 μg/mL, or less than 0.125 μg/mL.

An “effective amount” is an amount sufficient to effect beneficial or desired results. For example, a therapeutic amount is one that achieves the desired therapeutic effect. This amount can be the same or different from a prophylactically effective amount, which is an amount necessary to mitigate and/or prevent onset of disease or disease symptoms. An effective amount can be administered in one or more administrations, applications or dosages. A therapeutically effective amount of a composition depends on the composition selected. The compositions can be administered one or more times per day, or one or more times per week; including once every other day, once every day, twice per day, once every six hours, once every four hours, once every two hours, once every hour, etc. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments. For example, several divided doses may be administered at an appropriate time course such that of the compounds achieve the desired therapeutic result. A single vancomycin-teixobactin conjugate compound of a particular formula may be administered, or combinations of various a vancomycin-teixobactin conjugate compounds encompassing multiple formulas may be administered.

In a number of embodiments, a medicament comprising a vancomycin-teixobactin conjugate compound is administered in combination with an appropriate standard of care, such as the standard of care established by an appropriate agency (e.g., the United States Federal Drug Administration (FDA)). In many embodiments a medicament comprising a vancomycin-teixobactin conjugate compound is administered in combination with other antibiotic or immune stimulatory compounds, especially agency-approved compounds.

A number of embodiments of formulations of a medicament comprising a vancomycin-teixobactin conjugate compound are suggested for oral, topical, ocular, transdermal, transmucosal, parentenal, intranasal, pulmonary, epicutaneous, subcutaneous, intramuscular, or intravenous administration, as determined by various factors including compound bioavailability and the pathogen to be treated. As understood in the art, intravenous administration of vancomycin and teixobactin has generally been shown to have greater effectiveness over oral administration. One exception is for treatment of bacterial infections within the gastrointestinal tract in which oral administration has been shown to be effective. Topical administration has been shown to have efficacy against skin-related infections (e.g., MRSA). Other administration routes that directly target the bacterial infection have also been shown to be effective (e.g., ocular administration for ocular infection of MRSA).

Formulations may conveniently be presented in unit dosage form and may be prepared by any of the methods known in the art of pharmacy. Typically, these methods include the step of bringing into association a compound of at least one embodiment described herein, or a pharmaceutically acceptable salt or solvate thereof with the carrier which constitutes one or more accessory ingredients. An ocular drug delivery system may be utilized for delivery via ocular drops, ocular mist, ocular ointment, ocular gel, ocular cream, or any other appropriate system known in the art of pharmacy. An intranasal drug delivery system may be utilized for delivery via a nasal spray, nasal mist, nebulizer, or any other appropriate system known in the art of pharmacy. Likewise, a pulmonary drug delivery system may be utilized, for aerosol delivery via an inhaler, a nebulizer, dry powder, or any other appropriate system known in the art of pharmacy. An epicutaneous drug delivery system may be utilized for topical delivery via a cream, foam, gel, lotion, ointment, medicated patch, or any other appropriate system known in the art of pharmacy. A subcutaneous drug delivery system may be utilized for delivery via a syringe or any other appropriate system known in the art of pharmacy. An intramuscular drug delivery system may be utilized for delivery via a syringe or any other appropriate system known in the art of pharmacy. Further, an intravenous drug delivery system may be utilized, for fluid delivery via a syringe, infusion (e.g., drips), cannula, or any other appropriate system known in the art of pharmacy.

Various agents can be incorporated that improve the solubility of the various compounds described herein. For example, various compounds can be formulated with one or more adjuvants and/or pharmaceutically acceptable carriers according to the selected route of administration. For oral applications, gelatin, flavoring agents, or coating material can be added. In general, for solutions or emulsions, including for use in pulmonary applications, carriers may include aqueous or alcoholic/aqueous solutions, emulsions or suspensions, including saline and buffered media. Parenteral vehicles can include sodium chloride and potassium chloride, among others. In addition, intravenous vehicles can include fluid and nutrient replenishers, electrolyte replenishers and the like.

For IV applications, numerous fluids can be utilized in accordance with various embodiments. In some embodiments, a crystalloid or colloid solution is utilized. Crystalloid solutions include (but are not limited to) saline (i.e., NaCl 0.9%), lactated Ringer's, and Ringer's acetate. Colloid solutions include (but are not limited to) blood, albumin, and plasma. Medications can be administered in a continuous infusion, a secondary infusion, or a bolus.

Various embodiments of formulations also include at least one disintegrating agent, as well as diluent. In some embodiments, a disintegrating agent is a super disintegrant agent. One example of a diluent is a bulking agent such as a polyalcohol. In many embodiments, bulking agents and disintegrants are combined, such as, for example, PEARLITOL FLASH®, which is a ready to use mixture of mannitol and maize starch (mannitol/maize starch). In accordance with a number of embodiments, any polyalcohol bulking agent can be used when coupled with a disintegrant or a super disintegrant agent. Additional disintegrating agents include, but are not limited to, agar, calcium carbonate, maize starch, potato starch, tapioca starch, alginic acid, alginates, certain silicates, and sodium carbonate. Suitable super disintegrating agents include (but are not limited to) crospovidone, croscarmellose sodium, AMBERLITE (Rohm and Haas, Philadelphia, Pa.), and sodium starch glycolate.

In certain embodiments, diluents are selected from the group consisting of mannitol powder, spray dried mannitol, microcrystalline cellulose, lactose, dicalcium phosphate, tricalcium phosphate, starch, pregelatinized starch, compressible sugars, silicified microcrystalline cellulose, and calcium carbonate.

Several embodiments of a formulation further utilize other components and excipients. For example, sweeteners, flavors, buffering agents, and flavor enhancers to make the dosage form more palatable. Sweeteners include, but are not limited to, fructose, sucrose, glucose, maltose, mannose, galactose, lactose, sucralose, saccharin, aspartame, acesulfame K, and neotame. Common flavoring agents and flavor enhancers that may be included in the formulation of the present invention include, but are not limited to, maltol, vanillin, ethyl vanillin, menthol, citric acid, fumaric acid, ethyl maltol and tartaric acid.

Multiple embodiments of a formulation also include a surfactant. In certain embodiments, surfactants are selected from the group consisting of Tween 80, sodium lauryl sulfate, and docusate sodium.

Many embodiments of a formulation further utilize a binder. In certain embodiments, binders are selected from the group consisting of povidone (PVP) K29/32, hydroxypropylcellulose (HPC), hydroxypropylmethylcellulose (HPMC), ethylcellulose (EC), corn starch, pregelatinized starch, gelatin, and sugar.

Various embodiments of a formulation also include a lubricant. In certain embodiments, lubricants are selected from the group consisting of magnesium stearate, stearic acid, sodium stearyl fumarate, calcium stearate, hydrogenated vegetable oil, mineral oil, polyethylene glycol, polyethylene glycol 4000-6000, talc, and glyceryl behenate.

Preservatives and other additives, like antimicrobial, antioxidant, chelating agents, and inert gases, can also be present. (See generally, Remington's Pharmaceutical Sciences, 16th Edition, Mack, (1980), the disclosure of which is incorporated herein by reference.)

### EXAMPLES AND EXPERIMENTAL RESULTS

Biological data supports the biological activity and medicinal applications of the aforementioned vancomycin-teixobactin conjugates and analogues. Described within and in the attached are details of various vancomycin-teixobactin conjugates synthesized and tested for an inhibitory response on gram-positive bacteria. The data results of examples provided suggest that vancomycin-teixobactin conjugates have improved antibiotic activity against antibiotic-resistant bacteria, including VRE. These findings suggest that vancomycin-teixobactin conjugates are attractive drug candidates that circumvent the issues of antibiotic resistance.

It will be understood that the embodiments presented in this section are exemplary in nature and are provided to support and extend the broader disclosure. These embodiments are not meant to confine or otherwise limit the scope of the claims being sought.

### Example 1: MIC Assessment

Provided in FIGS. 9A to 9P are MIC assays of various vancomycin-teixobactin conjugates. These compounds assessed include Lys₁₀-teixo-vanco (FIG. 9A), Lys₁₀-teixo-(PEG4)-vanco (FIG. 9B), Lys₁₀-teixo-(PEG8)-vanco (FIG. 9C), Cbz-Lys₁₀-teixo₆₋₁₁-vanco (FIG. 9D), Lys₁₀-teixo₆₋₁₁-vanco (FIG. 9E), C8-Lys₁₀-teixo₆₋₁₁-vanco (FIG. 9F), C12-Lys₁₀-teixo₆₋₁₁-vanco (FIG. 9G), N-Me-D-Phe-Lys₁₀-teixo₆₋₁₁-vanco (FIG. 9H), Bph-Lys₁₀-teixo₆₋₁₁-vanco (FIG. 9I), Bph-Chg₆-Lys₁₀-teixo₇₋₁₁-vanco (FIG. 9J), C8-Chg₆-Lys₁₀-teixo₇₋₁₁-vanco (FIG. 9K), Benzoyl-Lys₁₀-teixo₅₋₁₁-vanco (FIG. 9L), Bph-Lys₁₀-teixo₇₋₁₁-vanco (FIG. 9M), Benzoyl-Lys₁₀-teixo₇₋₁₁-vanco (FIG. 9N), p-Chlorobenzoyl-Lys₁₀-teixo₇₋₁₁-vanco (FIG. 9O), Cbp-Lys₁₀-teixo₇₋₁₁-vanco (FIG. 9P), Cbp-Chg₁₁-Lys₁₀-teixo₇₋₁₁-vanco (FIG. 9Q), Cbp-Lys₁₀-teixo₇₋₁₁-6Ahx-vanco (FIG. 9R), Cbp-Orn₁₀-teixo₇₋₁₁-vanco (FIG. 9S), Cbp-aza-Lys₁₀-teixo₇₋₁₁-vanco (FIG. 9T), Cbp-Orn₁₀-Dap₉-teixo₇₋₁₁-vanco (FIG. 9U), Cbp-Lys₁₀-teixo₇₋₁₁-Hyn8-vanco (FIG. 9V), Cbp-Lys₁₀ (N₃)-teixo₇₋₁₁-propargylvanco (FIG. 9W), Cbp-Pra₁₀-teixo₇₋₁₁-azidobutylvanco (FIG. 9X), m,p-Dichlorobenzoyl-Lys₁₀-teixo₇₋₁₁-vanco (FIG. 9Y), and o,p-Dichlorobenzoyl-Lys₁₀-teixo₇₋₁₁-vanco (FIG. 9Z).

As can be seen within the data, these vancomycin-teixobactin conjugates have synergistic effect against Gram-positive bacteria that results in a MIC better than mixtures of teixobactin with vancomycin. Most vancomycin-teixobactin conjugates had a stronger antibiotic effect against VRE than vancomycin, including Lys₁₀-teixo-vanco, Lys₁₀-teixo-(PEG4)-vanco, Lys₁₀-teixo-(PEG8)-vanco, Cbz-Lys₁₀-teixo₆₋₁₁-vanco, C8-Lys₁₀-teixo₆₋₁₁-vanco, C12-Lys₁₀-teixo₆₋₁₁-vanco, N-Me-d-Phe-Lys₁₀-teixo₆₋₁₁-vanco, Bph-Lys₁₀-teixo₆₋₁₁-vanco, Bph-Chg₆-Lys₁₀-teixo₇₋₁₁-vanco, C8-Chg₆-Lys₁₀-teixo₇₋₁₁-vanco, Bph-Lys₁₀-teixo₇₋₁₁-vanco, Benzoyl-Lys₁₀-teixo₇₋₁₁-vanco, p-Chlorobenzoyl-Lys₁₀-teixo₇₋₁₁-vanco, Cbp-Lys₁₀-teixo₇₋₁₁-vanco, Cbp-Chg₁₁-Lys₁₀-teixo₇₋₁₁-vanco, Cbp-Lys₁₀-teixo₇₋₁₁-6Ahx-vanco, Cbp-Orn₁₀-teixo₇₋₁₁-vanco, Cbp-aza-Lys₁₀-teixo₇₋₁₁-vanco, Cbp-Orn₁₀-Dap₉-teixo₇₋₁₁-vanco, Cbp-Lys₁₀-teixo₇₋₁₁-Hyn8-vanco, Cbp-Lys₁₀ (N₃)-teixo₇₋₁₁-propargylvanco, Cbp-Pra₁₀-teixo₇₋₁₁-azidobutylvanco, m,p-Dichlorobenzoyl-Lys₁₀-teixo₇₋₁₁-vanco, and o,p-Dichlorobenzoyl-Lys₁₀-teixo₇₋₁₁-vanco. As expected, Lys₁₀-teixobactin had a strong effect against VRE, but Lys₁₀-teixobactin has limited utility in the clinic due to solubility issues. Cbp-Lys₁₀-teixo₇₋₁₁-vanco had as good of an antibiotic effect against VRE as teixobactin. Notably, Lys₁₀-teixo-vanco, Lys₁₀-teixo-(PEG4)-vanco, Lys₁₀-teixo-(PEG8)-vanco, Bph-Chg₆-Lys₁₀-teixo₇₋₁₁-vanco, C8-Chg₆-Lys₁₀-teixo₇₋₁₁-vanco, Bph-Lys₁₀-teixo₇₋₁₁-vanco, p-Chlorobenzoyl-Lys₁₀-teixo₇₋₁₁-vanco, C12-Lys₁₀-teixo₆₋₁₁-vanco, Cbp-Lys₁₀-teixo₇₋₁₁-vanco Cbp-Chg₁₁-Lys₁₀-teixo₇₋₁₁-vanco, Cbp-Lys₁₀-teixo₇₋₁₁-6Ahx-vanco, Cbp-Orn₁₀-teixo₇₋₁₁-vanco, Cbp-aza-Lys₁₀-teixo₇₋₁₁-vanco, Cbp-Orn₁₀-Dap₉-teixo₇₋₁₁-vanco, Cbp-Lys₁₀-teixo₇₋₁₁-Hyn8-vanco, Cbp-Lys₁₀ (N₃)-teixo₇₋₁₁-propargylvanco, Cbp-Pra₁₀-teixo₇₋₁₁-azidobutylvanco, and m,p-Dichlorobenzoyl-Lys₁₀-teixo₇₋₁₁-vanco have MIC values of lower than 16 μg/mL.

Various experiments and data, including antimicrobial activities of various vancomycin-teixobactin conjugates and mouse studies showing tolerability in vivo are attached. Compound SLP-02-094 used in the mouse studies is Cbp-Lys₁₀-teixo₇₋₁₁-vanco.

### Example 2: In vivo Assessments

A mouse tolerability study was conducted to ascertain adverse reactions. Female CD-1 mice were intravenously dosed with 20 mg/kg of Cbp-Lys₁₀-teixo₇₋₁₁-vanco or clovibactin at 0 hours and 12 hours through tail vein injection. Body weights and dose volumes were recorded. Mice receiving Cbp-Lys₁₀-teixo₇₋₁₁-vanco displayed no adverse reactions. Mice receiving clovibactin appeared lethargic with redness in their ear, feet and tail. Body weights were normal. The mice tolerated the teixobactin-vancomycin conjugate with no issues, and better than clovibactin.

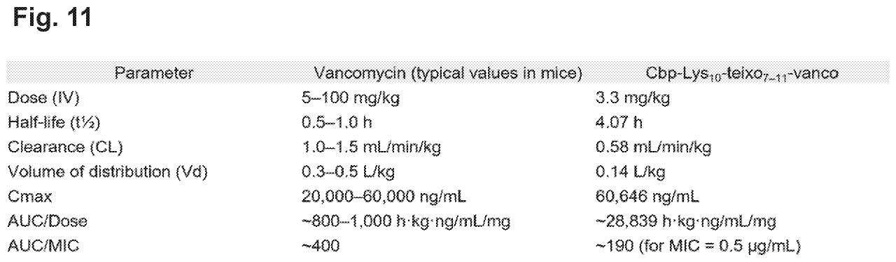

A mouse pharmacokinetics study was conducted on male ICR mice. Cbp-Lys₁₀-teixo₇₋₁₁-vanco was administered and dosed with each mouse follows: 1) intraperitoneal (IP) injection at a dose of 10 mg/kg; or 2) intravenous (IV) delivery at a dose of 3.3 mg/kg. Both routes of administration had no adverse effects and the mice remained healthy. Compared with vancomycin, Cbp-Lys₁₀-teixo₇₋₁₁-vanco significantly outperformed having a 4- to 8-times greater half-life and remained circulating for much longer (FIG. 11).

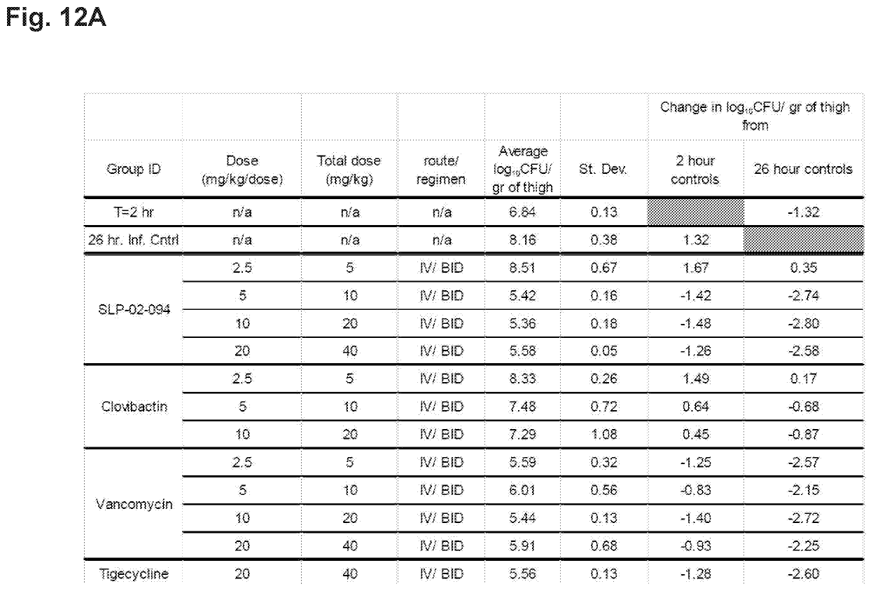

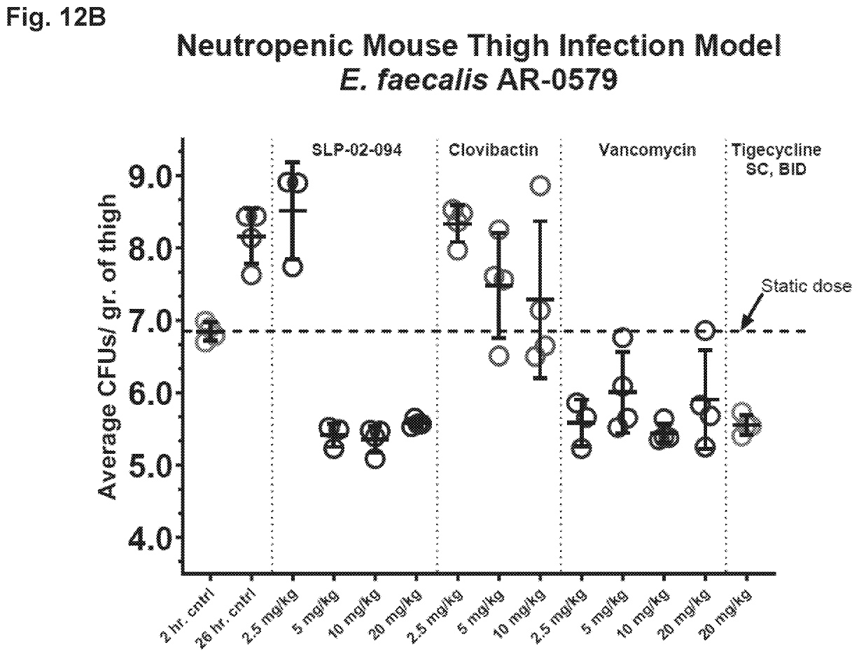

An efficacy study was conducted to ascertain antibiotic effects of the teixobactin-vancomycin conjugates on gram-positive bacteria. Female CD-1 mice were administered cyclophosphamide to induce neutropenia and then infected with gram-positive bacterial species E. faecalis (strain CDC AR-0679). Each mouse was infected with an inoculum of 1.5×10⁶ CFU in the thigh. Ater 2 and 14 hours post infection, groups of 4 mice each were dosed with Cbp-Lys₁₀-teixo₇₋₁₁-vanco, clovibactin, vancomycin, or tigecycline (FIG. 12A). At 2 and 26 hours post infection groups of 4 mice were euthanized. The right thighs from each animal were aseptically explanted, weighed, and homogenized to a uniform consistency. Homogenized samples were serially diluted and plated on bacterial growth media for CFU determination. CFUs were enumerated after overnight incubation at 37° C. The CFUs were adjusted for dilution and tissue weight for each thigh with averages and standard deviation calculated for each group.

At time of initial treatment, infection controls demonstrated a bioload of 6.84 log 10 CFUs/gram of thigh (FIG. 12A). The 26-hour infection controls demonstrated an increase in bioload of 1.32 log 10 CFUs from initiation of treatment. Test agent Cbp-Lys₁₀-teixo₇₋₁₁-vanco demonstrated a dose response with animals receiving 5, 10 or 20 mg/kg, resulting in CFU reductions from the 26-hour infection controls of 2.74, 2.80, and 2.58 log 10 CFUs, respectively (FIGS. 11A and 11B). Additionally, animals also demonstrated CFU reductions from the start of treatment ranging from 1.26 to 1.48 log 10 CFUs, suggesting active bacterial killing. Clovibactin delivered at 5 or 10 mg/kg demonstrated a slight reduction in CFUs of 0.68 and 0.87 log 10 CFUs from the 26-hour infection controls. Vancomycin and tigecycline both demonstrated CFU reductions from the start of treatment, as expected against this strain of gram-positive bacteria (FIGS. 12A and 12B).

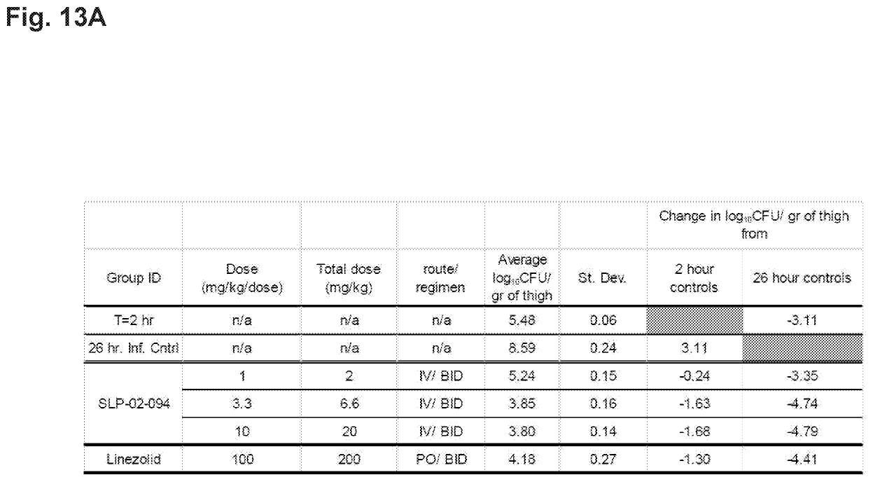

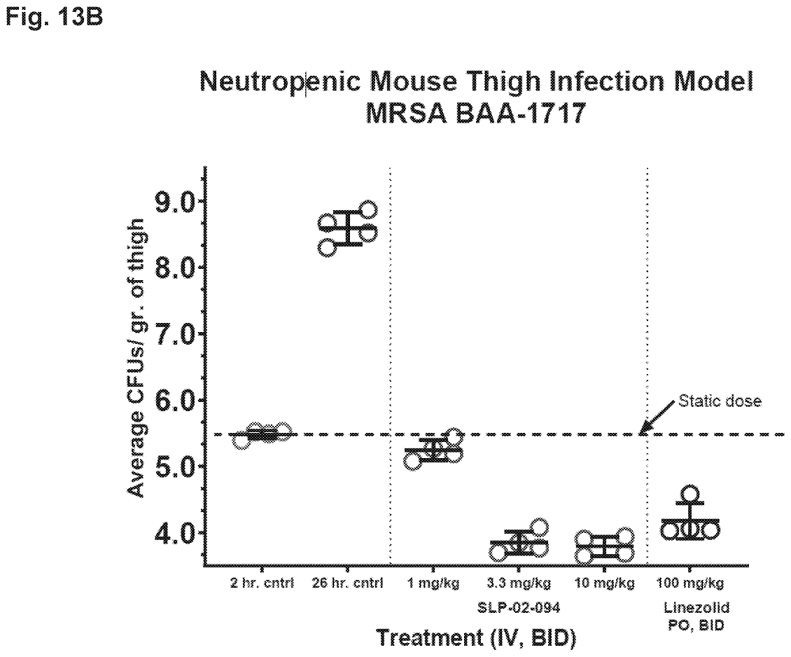

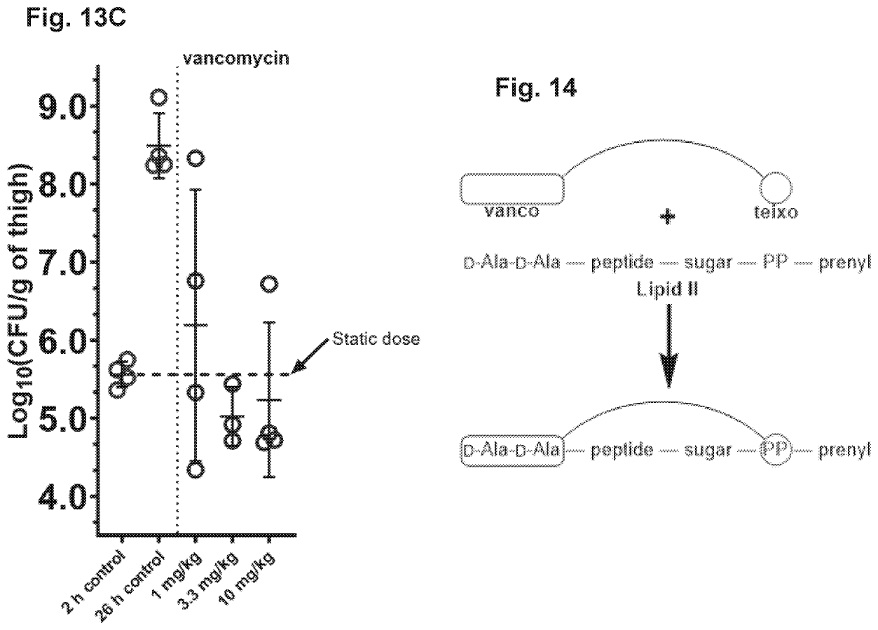

A second efficacy study was conducted to ascertain antibiotic effects of the teixobactin-vancomycin conjugates on difficult-to-treat gram-positive bacteria. Female CD-1 mice were administered cyclophosphamide to induce neutropenia and then infected with gram-positive bacterial species methicillin-resistant S. aureus (MRSA strain BAA-171). Each mouse was infected with an inoculum of 1.8×10⁶ CFU in the thigh. Ater 2 and 14 hours post infection, groups of 4 mice each were dosed with Cbp-Lys₁₀-teixo₇₋₁₁-vanco or linezolid (FIG. 13A). At 2 and 26 hours post infection groups of 4 mice were euthanized. The right thighs from each animal were aseptically explanted, weighed, and homogenized to a uniform consistency. Homogenized samples were serially diluted and plated on bacterial growth media for CFU determination. CFUs were enumerated after overnight incubation at 37° C. The CFUs were adjusted for dilution and tissue weight for each thigh with averages and standard deviation calculated for each group.

At time of initial treatment, infection controls demonstrated a bioload of 5.48 log 10 CFUs/gram of thigh (FIG. 13A). The 26-hour infection controls demonstrated an increase in bioload of 3.11 log 10 CFUs from initiation of treatment. Test agent Cbp-Lys₁₀-teixo₇₋₁₁-vanco demonstrated a dose response with animals receiving 1, 3.3 or 10 mg/kg BID resulting in CFU reductions from the 26-hour infection controls of 3.35, 4.74, and 4.79 log 10 CFUs, respectively. Additionally, animals also demonstrated CFU reductions from the start of treatment ranging from 0.24 to 1.68 log 10 CFUs, suggesting active bacterial killing. The positive control linezolid was delivered via oral gavage and demonstrated a CFU reduction from the 26-hour infection controls. (FIGS. 13A and 13B). The Cbp-Lys₁₀-teixo₇₋₁₁-vanco performed 10-fold better than vancomycin against the MRSA strain at the 3.3 and 10 mg/kg doses (FIG. 13C).

### Example 3: Experimental Analysis of Vancomycin-Teixobactin Conjugates

Vancomycin has been an important antibiotic of last resort since its clinical introduction in 1958. It is highly effective against Gram-positive pathogens such as MSSA and MRSA and typically has minimum inhibitory concentrations (MICs) of ≤2 μg/mL. Nevertheless, resistance is rising, with vancomycin-resistant pathogens such as vancomycin-resistant Enterococci (VRE) now widespread and vancomycin-resistant strains of S. aureus (VRSA) increasingly prevalent. Vancomycin binds to the d-Ala-d-Ala group in lipid II, inhibiting peptidoglycan cell-wall biosynthesis and ultimately leading to cell death. Bacteria have evolved resistance to vancomycin by mutating the d-Ala-d-Ala group in lipid II to d-Ala-d-Lac, which disrupts a key hydrogen bond with vancomycin and lipid II, resulting in a 1000-fold decrease in its binding affinity.

Teixobactin was first reported in 2015 as a promising antibiotic against Gram-positive pathogens. Teixobactin is an 11-residue cyclodepsipeptide, consisting of a macrolactone ring comprising residues 8-11 and an amphiphilic tail comprising residues 1-7. Teixobactin binds to the pyrophosphate group of lipid II and related cell-wall precursors, inhibits cell-wall biosynthesis, and ultimately kills bacteria by aggregating on the cell surface and lysing the bacteria. The ring of teixobactin binds the pyrophosphate group, while the amphiphilic tail of teixobactin interacts with the lipid bilayer membrane and drives aggregation by forming antiparallel β-sheets with additional teixobactin molecules. Gram-positive bacteria cannot develop resistance to teixobactin, because teixobactin targets the immutable pyrophosphate group of lipid II and related cell-wall precursors. Teixobactin is exceptionally potent, with MICs of 0.005-0.5 μg/mL against many Gram-positive bacteria, including MRSA, VRE, and Streptococcus pneumoniae.

Modification of vancomycin can restore activity against resistant pathogens and also impart new activity. A notable modification is the incorporation of an amidine residue on the peptide backbone of vancomycin to restore the key hydrogen-bond interaction with lipid II in vancomycin-resistant strains. Coupling guanidinium or tetraalkylammonium moieties to the C-terminus of vancomycin can increase its membrane permeability. Other promising conjugates of vancomycin have appendages of arginine and other guanidinium containing groups extending from the C-terminus of vancomycin. A number of these conjugates have shown increased activity against biofilms and persister cells formed by Gram-positive pathogens.

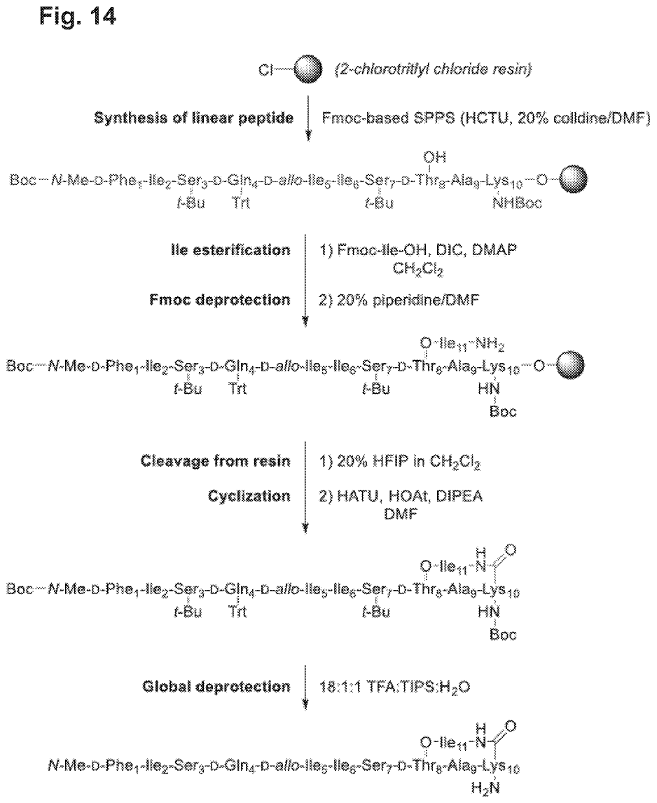

In the current study, we set out to determine whether we could enhance the activity of vancomycin and overcome resistance by conjugating it to teixobactin or a minimal fragment of teixobactin that targets the pyrophosphate group of lipid II and related cell wall precursors. We envisioned the conjugates binding to both the d-Ala-d-Ala and the pyrophosphate groups of lipid II. This concept is illustrated in FIG. 14. We have previously found that analogues of teixobactin in which the native allo-enduracididine residue at position 10 is replaced with lysine exhibit good antibiotic activity, albeit less than teixobactin. We thus hypothesized that conjugating vancomycin to Lys₁₀-teixobactin would result in enhanced activity over that of the individual components. We further envisioned that binding of the pyrophosphate group by the teixobactin component might help restore the diminished affinity of vancomycin for d-Ala-d-Lac in VRE.

Analogues of teixobactin in which residues 1-5 are replaced with a lipophilic group exhibit partial antibiotic activity. We hypothesized that conjugation of vancomycin to Lys₁₀-teixobactin analogues in which the tail is replaced with lipophilic groups might be sufficient to achieve enhanced activity. Here, we report that conjugation of vancomycin to the minimal pharmacophore of teixobactin results in antibiotics with excellent antibiotic activity against MSSA and MRSA and good activity against VRE.

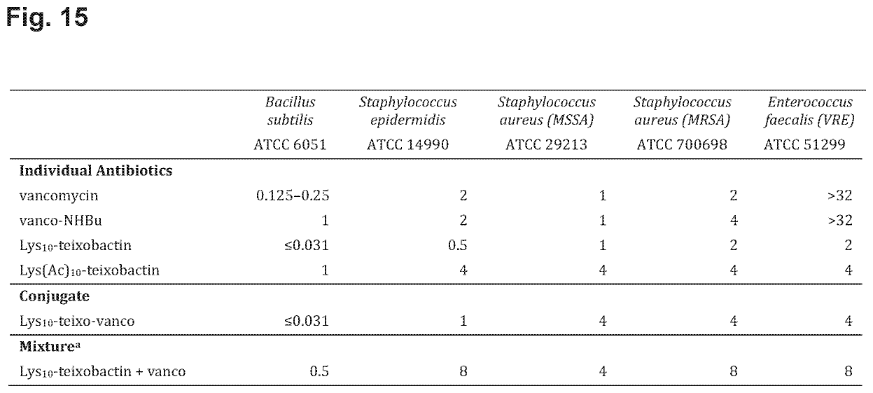

We prepared vancomycin-teixobactin conjugates by EDC coupling of vancomycin to Lys₁₀-teixobactin and truncated teixobactin analogues (FIG. 15). Coupling of Lys₁₀-teixobactin occurs exclusively at the amino group of the lysine side chain, without reaction at the N-terminal amino group of the N-methyl-d-phenylalanine residue. We then assessed the antibiotic activity of the conjugates through MIC assays against a panel of Gram-positive bacteria. We used the Gram-negative bacteria E. coli as a negative control for the MIC assays. We performed the MIC assays in the presence of 0.002% polysorbate 80, which helps prevent teixobactin derivatives from being adsorbed by the plasticware used for the assays.

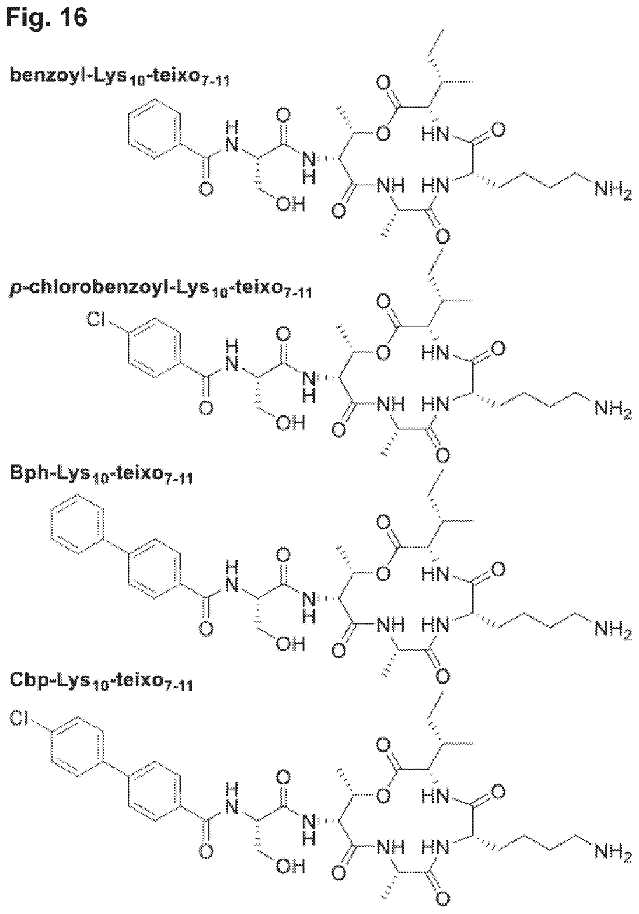

The conjugate of vancomycin and Lys₁₀-teixobactin, Lys₁₀-teixo-vanco, is active, exhibiting MICs of 4 μg/mL against both MRSA and VRE (FIG. 16). These values are noteworthy because acylation of Lys₁₀-teixobactin decreases its antibiotic activity—Lys(Ac)₁₀-teixobactin has an MIC value of 4 μg/mL against both MRSA and VRE, while Lys₁₀-teixobactin has an MIC value of 2 μg/mL. The MIC value of Lys₁₀-teixo-vanco against VRE is encouraging, because vancomycin is not active against VRE and the molar concentration of Lys₁₀-teixo-vanco at 4 μg/mL (1.3 μM) is less than the molar concentration of Lys(Ac)₁₀-teixobactin at 4 μg/mL (2.9 μM). Furthermore, Lys₁₀-teixo-vanco is more active than a mixture of equal weights of Lys₁₀-teixobactin and vancomycin, which exhibits MICs of 8 μg/mL against MRSA and VRE. This control experiment demonstrates that synergy is not achieved by mixing the two antibiotics and that mixing the two components without covalent conjugation may be detrimental to antibiotic activity.

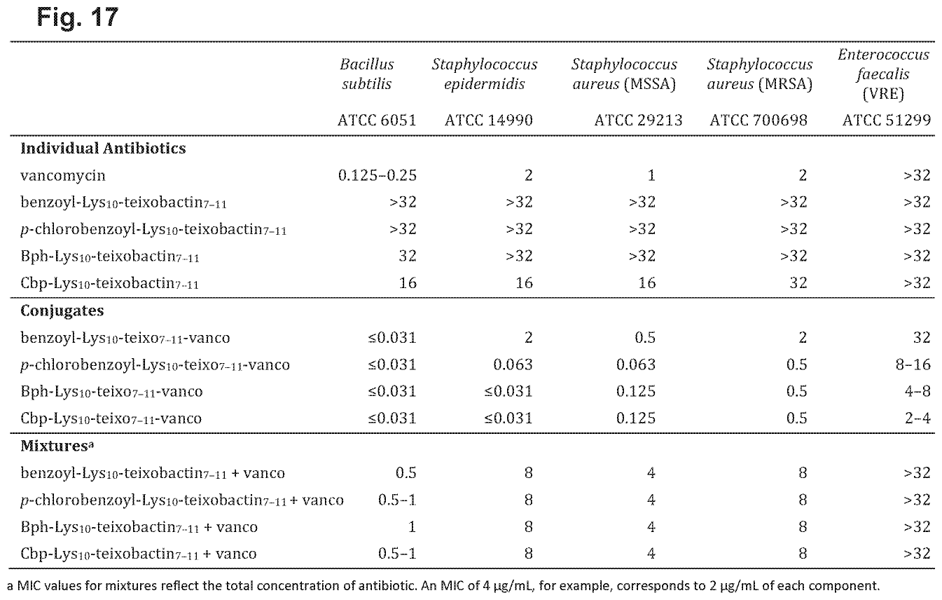

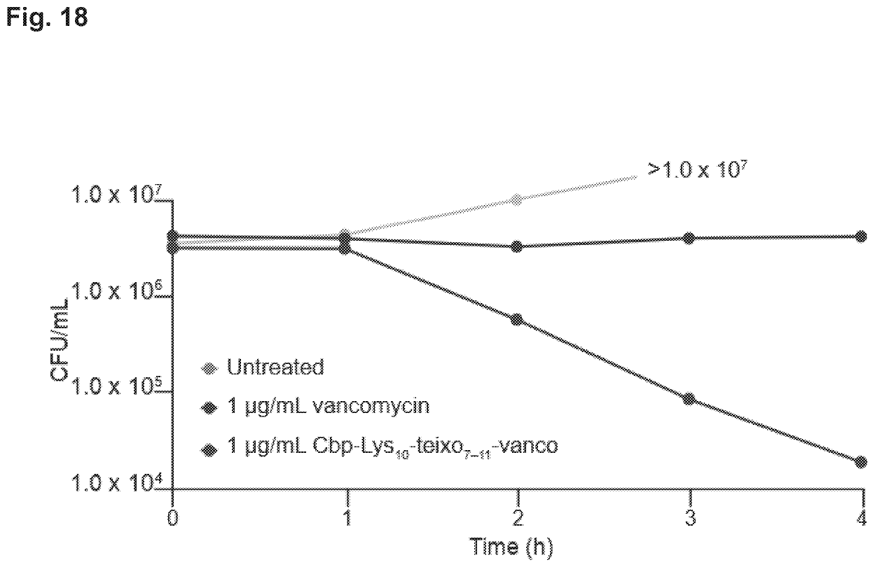

The modest enhancement of vancomycin activity by conjugation to full-length Lys₁₀-teixobactin prompted us to explore truncated analogues of Lys₁₀-teixobactin in which the hydrophobic region of the tail (residues 1-6) were replaced with a lipophilic group. We thus prepared vancomycin-teixobactin conjugates to benzoyl-Lys₁₀-teixobactin₇₋₁₁ and p-chlorobenzoyl-Lys₁₀-teixobactin₇₋₁₁ and the corresponding biphenyl analogues Bph-Lys₁₀-teixobactin₇₋₁₁ and Cbp-Lys₁₀-teixobactin₇₋₁₁ (FIG. 17). These truncated Lys₁₀-teixobactin analogues show little or no activity against Gram-positive bacteria (FIG. 18).

Conjugation of vancomycin to benzoyl-Lys₁₀-teixobactin₇₋₁₁ results in a modest yet promising increase in activity over vancomycin against several of the Gram-positive bacteria. Notably, the MIC of benzoyl-Lys₁₀-teixo₇₋₁₁-vanco against MSSA is 0.5 μg/mL, while that of vancomycin is 1 μg/mL; and the MIC against B. subtilis is <0.031 μg/mL, while that of vancomycin is 0.125-0.25 μg/mL. For MRSA and S. epidermidis, the activity of the conjugate is equal to that of vancomycin. The conjugate shows modest activity against VRE (32 μg/mL), while vancomycin is inactive.

The conjugates of vancomycin with p-chlorobenzoyl-Lys₁₀-teixobactin₇₋₁₁, Bph-Lys₁₀-teixobactin₇₋₁₁, and Cbp-Lys₁₀-teixobactin₇₋₁₁ exhibit substantially enhanced activity against all of the Gram-positive bacteria tested, including VRE (FIG. 18). p-Chlorobenzoyl-Lys₁₀-teixo₇₋₁₁-vanco is substantially more active than benzoyl-Lys₁₀-teixo₇₋₁₁-vanco, with activities of 0.063 μg/mL against MSSA, 0.5 μg/mL against MRSA, and 8-16 μg/mL against VRE. The biphenyl analogue Bph-Lys₁₀-teixo₇₋₁₁-vanco is also more active than benzoyl-Lys₁₀-teixo₇₋₁₁-vanco, with activities of 0.125 μg/mL against MSSA, 0.5 μg/mL against MRSA, and 4-8 μg/mL against VRE. The p-chlorobiphenyl analogue Cbp-Lys₁₀-teixo₇₋₁₁-vanco exhibits greater activity against VRE than Bph-Lys₁₀-teixo₇₋₁₁-vanco (2-4 μg/mL vs. 4-8 μg/mL) and is equally active against MSSA and MRSA (0.125 μg/mL and 0.5 μg/mL). Collectively, these results demonstrate that conjugation of two components that target different regions of lipid II result in enhanced antibiotic activity.

We performed a time-kill assay against MRSA with Cbp-Lys₁₀-teixo₇₋₁₁-vanco, to assess whether this conjugate is bactericidal. We performed this assay by culturing MRSA in the presence of the conjugate at 1 μg/mL (2×MIC) and determined the concentration of colony-forming units (CFU) as a function of time, and we compared the conjugate to vancomycin (1 μg/mL) and no antibiotic. Over the course of four hours, the concentration of bacteria decreased by three log 10 units in the presence of the conjugate (FIG. 19). In contrast, the concentration of bacteria increased three-fold in the presence of vancomycin, and the bacteria grew rapidly in the absence of antibiotic. These results show that Cbp-Lys₁₀-teixo₇₋₁₁-vanco is bactericidal at concentrations above the MIC.

To determine the effect of the truncated Lys₁₀-teixobactin conjugates on mammalian cells, we assessed their hemolytic activity and cytotoxicity against human red blood cells and HEK-293 cells. Although none of these compounds proved highly lytic or toxic, they do exhibit increasing hemolytic activity and cytotoxicity with increasing hydrophobicity of the aromatic amide group. Benzoyl-Lys₁₀-teixo₇₋₁₁-vanco and p-chlorobenzoyl-Lys₁₀-teixo₇₋₁₁-vanco both exhibit no hemolytic activity at concentrations as high as 100 μg/mL and no cytotoxicity at concentrations as high at 50 UM (113 and 115 μg/mL). Bph-Lys₁₀-teixo₇₋₁₁-vanco exhibits no hemolytic activity at concentrations as high as 50 g/mL and slight hemolytic activity (2%) at 100 μg/mL, as well as no cytotoxicity at concentrations as high as 25 μM (59 μg/mL). Cbp-Lys₁₀-teixo₇₋₁₁-vanco exhibits no hemolytic activity at concentrations as high as 25 μg/mL and slight hemolytic activity (4%) at 100 μg/mL, as well as no cytotoxicity at concentrations as high as 6.25 μM (15 μg/mL) and slight cytotoxicity at 12.5 μM (30 μg/mL). The far greater activity of the conjugates against bacteria over mammalian cells suggests that they may be good candidates for preclinical in vivo studies in which the antibiotic is administered intravenously and evaluated for efficacy.

In summary, conjugation of vancomycin to the macrocyclic pharmacophore of teixobactin significantly enhances antibiotic activity against Gram-positive bacteria compared to the components alone and mixtures of the two components. Lys₁₀-teixobactin analogues containing the minimal pharmacophore of the macrolactone ring and an aromatic amide in place of the aggregation-prone peptide tail show little or no activity on their own. Coupling vancomycin to this minimal pharmacophore creates a conjugate with binding sites for both the d-Ala-d-Ala group and the pyrophosphate group of lipid II. Even though the d-Ala-d-Ala group is mutated to d-Ala-d-Lac in VRE, conjugating the two inactive components imparts respectable antibiotic activity against this problematic pathogen. Notably, mixtures of vancomycin and minimal teixobactin pharmacophores do not show enhanced activity against any of the Gram-positive bacteria tested.

### Materials and Methods

### General Information.

All chemicals were used as received unless otherwise noted. Reagent-grade solvents, chemicals, amino acids, and resin were used as received, with the following three exceptions: (1) Methylene chloride (CH₂Cl₂) was passed through neutral alumina under argon prior to use. (2) Amine-free N,N-dimethylformamide (DMF) was dried through an alumina and amine scavenger resin column under argon prior to use. (3) Vancomycin (HCl) was purified through reverse-phase HPLC (RP-HPLC) prior to use. Solid-phase peptide synthesis was carried out manually in a 25-mL coupling vessel. Preparative RP-HPLC was performed on a instrument equipped with a SB-C18 column. Analytical reverse-phase HPLC was performed with an 2.6 u XB-C18 column. UV detection (214 nm) was used for both preparative and analytical HPLC. HPLC grade acetonitrile (CH₃CN) and deionized water (18 MΩ), each containing 0.1% trifluoroacetic acid (TFA), were used as solvents for both preparative and analytical reverse-phase HPLC. HPLC grade acetonitrile (CH₃CN) and deionized water (18 MΩ), each containing 0.1% trifluoroacetic acid (TFA), were used for analytical and preparative reverse-phase HPLC. All peptides were prepared and used as trifluoroacetate salts and were assumed to have one trifluoracetic acid molecule per amine group on each peptide.

### Representative Synthesis of Teixobactin Analogues.

### Loading and Capping of 2-Chlorotrityl Resin.

2-chlorotrityl resin (300 mg, 1.42 mmol/g) was added to a 10-mL Poly-Prep chromatography column. The resin was swelled in dry CH₂Cl₂ (10 mL) for 30 min. The resin was drained and a solution of Fmoc-Lys (Boc)-OH (134 mg, mmol, equiv) in dry CH₂Cl₂ (5 mL) and 2,4,6-collidine (300 μL) was added and suspension rocked for 6 h. The solution was then drained and a solution of dry CH₂Cl₂ (5 mL), MeOH (0.8 mL) and DIPEA (0.4 mL) was added to the Poly-Prep column. The column was rocked for 1 h to cap the unreacted 2-chlorotrityl chloride resin sites. The solution was then drained, and the resin was washed with dry CH₂Cl₂ (3×5 mL).

### Coupling of Linear Peptide.

The linear peptide was synthesized from C-terminus to N-terminus through the following cycles: coupling of amino acid (0.680 mmol, 4.0 equiv) with HCTU (0.680 mmol, 4.0 equiv) in 20% 2,4,6-collidine in DMF (4 mL) for 30 min, resin washing with DMF (8×4 mL), Fmoc-deprotection with 20% piperidine in DMF (4 mL) for 10 min, and resin washing with DMF (8×4 mL).

### Esterification of Fmoc-Ile-OH.

A solution of Fmoc-Ile-OH (547 mg, 1.55 mmol, 10.0 equiv), diisopropylcarbodiimide (240 mg, 1.55 mmol, 10.0 equiv) and dimethylaminopyridine (19 mg, 0.156 mmol, 1.0 equiv) in dry CH₂Cl₂ (5 mL) was added to the Poly-Prep column. The column was rocked for 1 h. The solution was drained and washed with dry CH₂Cl₂ (3×5 mL) and DMF (3×5 mL).

Fmoc deprotection of Ile₁₁.

20% piperidine (v/v) in dry DMF (5 mL) was added to the Poly-Prep column and rocked for 20 min. The solution was drained from the column and resin was washed with dry DMF (3×5 mL) then DCM (3×5 mL).

Cleavage from Resin.

A solution of 1,1,1,3,3,3-hexafluoroisopropanol (3.5 mL) and dry CH₂Cl₂ was made (11.5 mL). Half of the solution was added to the Poly-Prep column and placed on the rocker for 1 h. The solution was drained into a clean round bottom flask. The remaining half of the solution was added to the Poly-Prep column and rocked for 30 min. The solution was then added to the same round bottom flask. The resin was washed with CH₂Cl₂ (2×5 mL) and drained into the same round bottom flask. The filtrates were then concentrated under reduced pressure to afford the clear oil.

### Solution-phase Cyclization.

A solution of HATU (353 mg, 0.928 mmol, 6.00 equiv) and HOAt (126 mg, 0.926 mmol, 6.00 equiv) in dry DMF (125 mL) was added to the round bottom flask and stirred until the oil was dissolved. DIPEA (162 μL, 0.930 mmol, 6.00 equiv) was added to the solution and stirred under nitrogen for 12 h. The mixture was concentrated under reduced pressure to afford a yellow solid. The solid was placed under vacuum to remove any residual solvents.

### Global Deprotection and Ether Precipitation.

A solution of trifluoroacetic acid (TFA) (9 mL), triisopropylsilane (TIPS) (0.5 mL), and water (0.5 mL) was added to the round bottom flask containing the protected, cyclized peptide. The reaction was stirred under nitrogen for 1.5 h. After deprotection, equal volumes of the deprotected peptide solution were aliquoted into two 50-ml conical tubes containing ice cold diethyl ether where precipitation was observed. The precipitate was pelleted by centrifugation for 10 min at 2000×g. The ether supernatant was then decanted into a clean Erlenmeyer flask. This process was repeated 2×. The dried pellets were then dissolved in 20% CH₃CN in H₂O and water and lyophilized to afford an off-white/light yellow powder.

Reverse-Phase HPLC purification.

The filtered crude cyclic peptide was injected at 25% CH₃CN and eluted with a gradient of 25-40% CH₃CN with 0.1% TFA over 120 min. Collected fractions were analyzed by MALDI-TOF MS and analytical HPLC. Pure fractions were combined and concentrated by rotary evaporation. The concentrate was transferred to a 15 ml conical, frozen in liquid N₂, and lyophilized to afford a white powder.

| | Yield | | Calculated MW as |
|---|---|---|---|
| Conjugate | (mg) | % yield | TFA salt |
| Lys₁₀-teixobactin | 23.9 | 5 | (•2 TFA) |
| Lys₁₀(Ac)-teixobactin | 30.6 | 13 | (•1 TFA) |
| Benzoyl-Lys₁₀-teixo₇₋₁₁ | 74.2 | 43 | (•1 TFA) |
| p-chlorobenzoyl-Lys₁₀- | 46.6 | 36 | (•1 TFA) |
| teixo₇₋₁₁ | | | |
| Bph-Lys₁₀-teixo₇₋₁₁ | 56.8 | 31 | (•1 TFA) |
| Cbp-Lys₁₀-teixo₇₋₁₁ | 49.3 | 33 | (•1 TFA) |

### Representative Chemical Synthesis of Vancomycin Conjugates.

Vancomycin (TFA) (1.5 molar equiv) was added to an 10-mL round bottom flask containing a stir bar. HOAt (5.4 molar equiv) and EDC-HCl (5.1 molar equiv) were added to the vial. Dry DMF (12 mM) was added to the vial. The mixture was then stirred until all reagents were in solution. Lys₁₀-teixobactin (TFA) (1 molar equiv) was added to the reaction mixture. N-methylmorpholine (10% total volume) was added dropwise and the reaction mixture turned from cloudy white to cloudy yellow. The reaction was stirred at room temperature for 2.5-24 hours, at which point the reaction mixture turned clear. 1 mL HPLC grade H₂O was added slowly to dilute the reaction. The crude mixture was concentrated to dryness. The crude solid was redissolved in 5% CH₃CN in H₂O and purified by RP-HPLC on a semi-preparative C18 column with a gradient of 5 to 55% CH₃CN/H₂O with 0.1% TFA over 70 min. Collected fractions were analyzed by LC-MS and analytical HPLC. Pure fractions were combined and concentrated by rotary evaporation. The concentrate was transferred to a 15 ml conical, frozen in liquid N₂, and lyophilized to afford a white powder. The appropriate fractions were lyophilized, and the product was isolated as a TFA salt (4 mg, 18% yield). The compound was stored as a lyophilized, white powder at −20° C.

| | Yield | | Calculated MW as |
|---|---|---|---|
| Conjugate | (mg) | % yield | TFA salt |
| Lys10-teixo-vanco | 4.0 | 18 | 2989.85 (•3 TFA) |
| Vanco-NHBu | 2.0 | 11 | 1732.43 (•2 TFA) |
| Benzoyl-Lys₁₀-teixo₇₋₁₁- | 6.7 | 42 | 2264.00 (•2 TFA) |
| vanco | | | |
| p-chlorobenzoyl-Lys₁₀- | 8.7 | 41 | 2298.44 (•2 TFA) |
| teixo₇₋₁₁-vanco | | | |
| Bph-Lys₁₀-teixo₇₋₁₁-vanco | 5.1 | 47 | 2340.1 (•2 TFA) |
| Cbp-Lys₁₀-teixo₇₋₁₁-vanco | 10.2 | 58 | 2374.54 (•2 TFA) |

### MIC Assays of Teixobactin and Vancomycin Analogues.

All media used contained 0.002% polysorbate 80 to prevent the teixobactin from adhering to the 96-well microtiter plate.

Bacillus subtilis, Staphyloccocus epidermidis, Escherichia coli, Staphylococcus aureus, and Enterococcus faecalis were cultured from glycerol stocks in Mueller-Hinton Broth (B. sub, S. epi, E. coli, MSSA), Blood-Heart-Infusion media (MRSA) and Blood-Heart-Infusion media with 4 μg/mL of vancomycin (VRE). overnight in a shaking incubator at 37° C. After incubation, 200 μL aliquots of each bacterial culture was added to a single well in a 96-well plate. A plate reader was then used to determine the OD₆₀₀ of each culture. Based on previously reported colony-forming units (CFU) per milliliter of each bacterial strain, each overnight culture was diluted to 1×10⁶ CFU/mL with both media.

An aliquot of a 1 mg/mL peptide stock solution in DMSO was diluted with media to a 16 μg/mL of peptide in culture media. 200 μL aliquots of the 16 μg/mL peptide stock was transferred to a sterile, non-treated 96-well plate. Two-fold serial dilutions were made across a 96-well plate with media to achieve a final volume of 100 μL in each well. These solutions had the following concentrations: 64, 32, 16, 8, 4, 2, 1, 0.5, 0.25, 0.125, and 0.0625 ug/mL.

A 100 μL aliquot of the previously prepared 1×10⁶ CFU/mL bacterial solution was added to each of the 11 wells, resulting in a final bacterial concentration of 5×10⁵ CFU/mL and the following final concentrations of peptide: 32, 16, 8, 4, 2, 1, 0.5, 0.25, 0.125, 0.0625, and 0.03125 μg/mL. The plate was covered with a lid and incubated at 37° C. for 16 h. The OD₆₀₀ of each well was measured using a 96-well UV/Vis plate reader. The MIC values were taken as the lowest concentration of peptide that had no bacterial growth. Each MIC assay was run in quadruplicate. Several of the MIC assays were repeated to ensure reproducibility.

### Cell Culture.

HEK293 cell cultures were maintained in complete media of Eagle's Minimum Essential Medium (EMEM) supplemented with 10% heat-inactivated fetal bovine serum (FBS), 100 μg/mL penicillin and 100 μg/mL streptomycin at pH 7.4 in a humidified 5% CO₂ atmosphere at 37° C. using a Fischer Scientific Forma Serie 2 Water Jacketed CO₂ incubator. All experiment were performed in triplicate in sterile half-area 96-well plates that were cell culture treated.

### Plating Cells.

HEK293 cells were seeded at 2,000-2,500 cells per well in the inner 60 wells of 96-well half-area plates to a total volume of 50 μL using complete media. 100 μL of plain EMEM (no cells) was added to the outer wells of the plate. The plates were then incubated in a humidified 5% CO₂ atmosphere at 37° C. for 24 h after plating to allow for cells to adhere to the plate. The complete media was removed from the inner wells prior to treatment with peptide.

### Characterization of Vancomycin-N-Butylamide

### Structure of Vancomycin-N-Butylamide

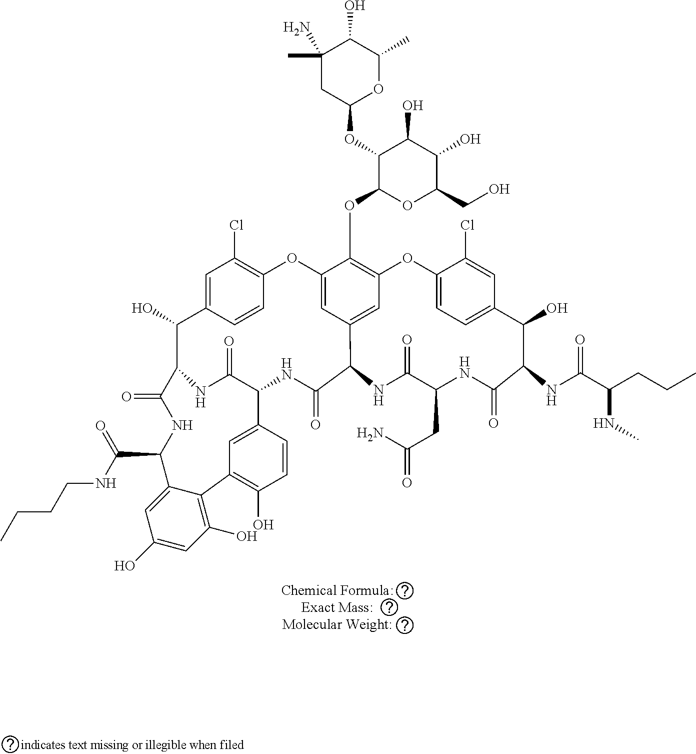

### Characterization of Lys(Ac)₁₀-Teixobactin

### Structure of Lys(Ac)₁₀-Teixobactin

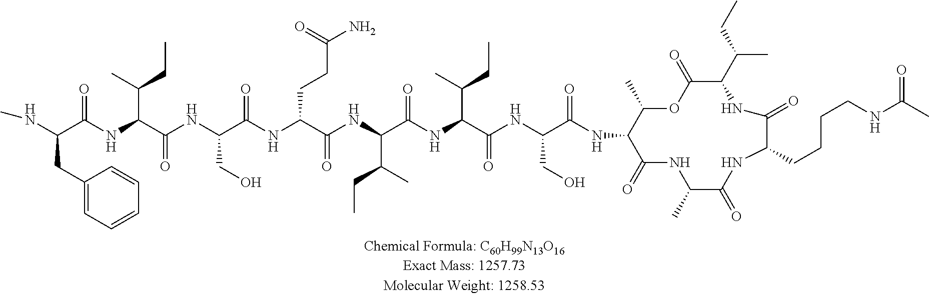 [SMILES: CC[C@H](C)[C@H](NC(=O)[C@@H](Cc1ccccc1)NC)C(=O)N[C@@H](CO)C(=O)N[C@H](CCC(N)=O)C(=O)N[C@@H](C(=O)N[C@H](C(=O)N[C@@H](CO)C(=O)N[C@H]1C(=O)N[C@@H](C)C(=O)N[C@@H](CCCCNC(C)=O)C(=O)N[C@@H]([C@@H](C)CC)C(=O)O[C@H]1C)[C@@H](C)CC)[C@@H](C)CC]

Characterization of Benzoyl-Lys₁₀-Teixobactin₇₋₁₁
Structure of Benzoyl-Lys₁₀-Teixobactin₇₋₁₁

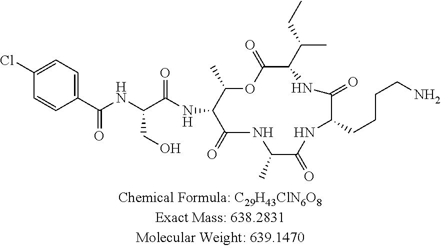 [SMILES: CC[C@H](C)[C@@H]1NC(=O)[C@H](CCCCN)NC(=O)[C@H](C)NC(=O)[C@H](NC(=O)[C@H](CO)NC(=O)c2ccc(Cl)cc2)[C@H](C)OC1=O]

Characterization of p-Chlorobenzoyl-Lys₁₀-Teixobactin₇₋₁₁
Structure of p-Chlorobenzoyl-Lys₁₀-Teixobactin₇₋₁₁

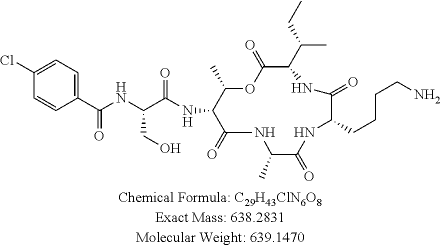 [SMILES: CC[C@H](C)[C@@H]1NC(=O)[C@H](CCCCN)NC(=O)[C@H](C)NC(=O)[C@H](NC(=O)[C@H](CO)NC(=O)c2ccc(Cl)cc2)[C@H](C)OC1=O]

Characterization of Bph-Lys₁₀-Teixobactin₇₋₁₁
Structure of Bph-Lys₁₀-Teixobactin₇₋₁₁

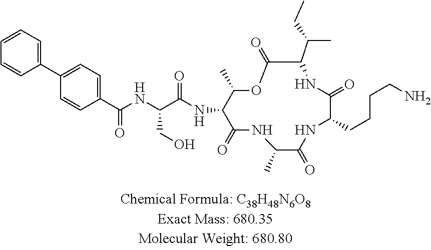

Characterization of Cbp-Lys₁₀-Teixobactin₇₋₁₁
Structure of Cbp-Lys₁₀-Teixobactin₇₋₁₁

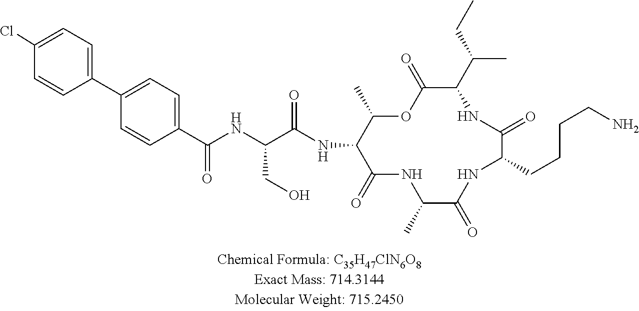 [SMILES: CC[C@H](C)[C@@H]1NC(=O)[C@H](CCCCN)NC(=O)[C@H](C)NC(=O)[C@H](NC(=O)[C@H](CO)NC(=O)c2ccc(-c3ccc(Cl)cc3)cc2)[C@H](C)OC1=O]

Characterization of Benzoyl-Lys₁₀-Teixobactin₇₋₁₁-vanco
Structure of Benzoyl-Lys₁₀-Teixobactin₇₋₁₁-vanco

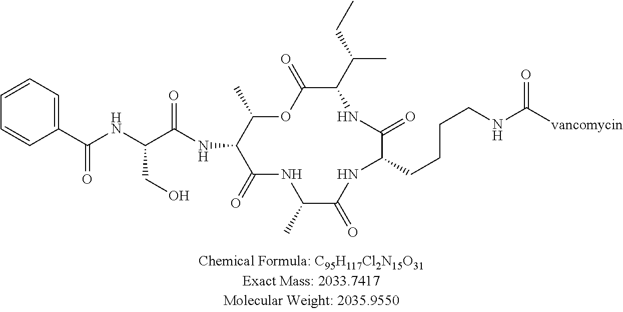

Characterization of p-Chlorobenzoyl-Lys₁₀-Teixobactin₇₋₁₁-vanco
Structure of p-Chlorobenzoyl-Lys₁₀-Teixobactin₇₋₁₁-vanco

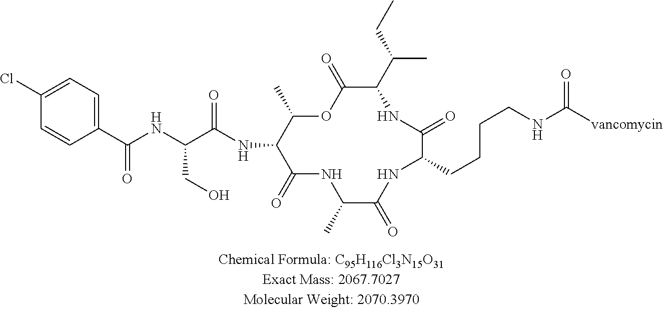

Characterization of Bph-Lys₁₀-Teixobactin₇₋₁₁-vanco
Structure of Bph-Lys₁₀-Teixobactin₇₋₁₁-vanco

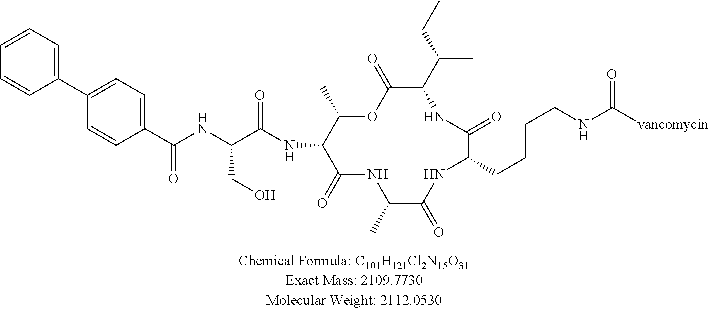

Characterization of Cbp-Lys₁₀-Teixobactin₇₋₁₁-vanco
Structure of Cbp-Lys₁₀-Teixobactin₇₋₁₁-vanco

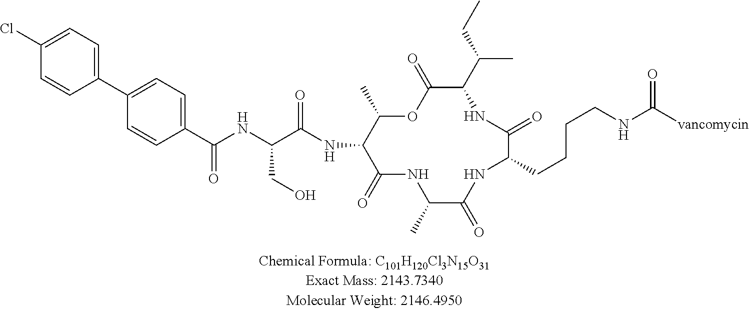

1. A composition comprising:
a teixobactin ring conjugated to a vancomycin.

2. The composition of claim 1, wherein the teixobactin ring comprises a substituted amino acid, wherein the teixobactin ring is conjugated to the vancomycin via its side chain.

3. The composition of claim 2, wherein the substituted amino acid an amino group, a triazole group, a carboxylate group, or a thiol for forming a bond utilized for conjugation to the vancomycin.

4. The composition of claim 1, wherein the teixobactin ring comprises the following structure: 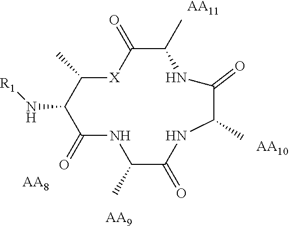
wherein X is O or NH;
wherein AAs is D-Thr, D-aza-Thr, or hydroxyasparagine;
wherein AA₉ is Ala, diamino propionic acid (Dap), 2,4-diamino butyric acid, Orn, Lys, 6-azidolysine, 2,7-diaminoheptanoic acid, 1,2,3-triazole-4-carboxamide, 1,2,4-triazole-3-carboxamide, propargylglycine, Asp, Glu, or Cys;
wherein AA₁₀ is allo-End, diamino propionic acid (Dap), 2,4-diamino butyric acid, Orn, Lys, 6-azidolysine, 2,7-diaminoheptanoic acid, 1,2,3-triazole-4-carboxamide, 1,2,4-triazole-3-carboxamide, propargylglycine, Asp, Glu, or Cys;
wherein AA₁₁ is Ile, Val, Leu, Chg, or other amino acid with bulky hydrophobic side chain;
wherein R¹ is H or a teixobactin tail comprising one or more amino acids.

5. The composition of claim 4, wherein the teixobactin ring is conjugated to the vancomycin via a sidechain of AA₁₀.

6. The composition of claim 4, wherein the teixobactin ring is conjugated to the vancomycin via a sidechain of AA₉.

7. The composition of claim 4, wherein the teixobactin tail comprises the following structure: 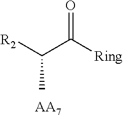
wherein R² is H, a methyl group, N-Me-d-Phe, an alkyl group C2 to C12, a benzyloxycarbonyl group, a benzoyl group, a halobenzoyl group, a biphenyl group, a halobiphenyl group, or an N-linkage that conjugates the vancomycin to the teixobactin tail
wherein AA₇ is Ser, Thr, allo-Thr, or other amino acid with beta hydroxy group;
wherein Ring is the teixobactin ring.

8. The composition of claim 4, wherein the teixobactin tail comprises the following structure: 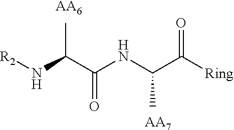
wherein R² is H, a methyl group, N-Me-d-Phe, an alkyl group C2 to C12, a benzyloxycarbonyl group, a benzoyl group, a halobenzoyl group, a biphenyl group, a halobiphenyl group, or an N-linkage that conjugates the vancomycin to the teixobactin tail;
wherein AA₆ is Ile, Val, Leu, Chg, or other amino acid with bulky hydrophobic side chain;
wherein AA₇ is Ser, Thr, allo-Thr, or other amino acid with beta hydroxy group;
wherein Ring is the teixobactin ring.

9. The composition of claim 4, wherein the teixobactin tail comprises the following structure: 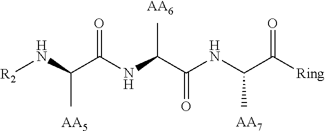
wherein R² is H, a methyl group, N-Me-d-Phe, an alkyl group C2 to C12, a benzyloxycarbonyl group, a benzoyl group, a halobenzoyl group, a biphenyl group, a halobiphenyl group, or an N-linkage that conjugates the vancomycin to the teixobactin tail;
wherein AA₅ is D-allo-Ile, or any other D- or L-amino acid with hydrophobic side chain;
wherein AA₆ is Ile, Val, Leu, Chg, or other amino acid with bulky hydrophobic side chain;
wherein AA₇ is Ser, Thr, allo-Thr, or other amino acid with beta hydroxy group;
wherein Ring is the teixobactin ring.

10. The composition of claim 4, wherein the teixobactin tail comprises the following structure: 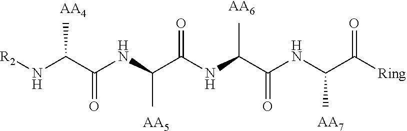
wherein R² is H, a methyl group, N-Me-d-Phe, an alkyl group C2 to C12, a benzyloxycarbonyl group, a benzoyl group, a halobenzoyl group, a biphenyl group, a halobiphenyl group, or an N-linkage that conjugates the vancomycin to the teixobactin tail;
wherein AA₄ is D-Gln, any other D-amino acid, diamino propionic acid (Dap), 2,4-diamino butyric acid, Orn, Lys, 6-azidolysine, 2,7-diaminoheptanoic acid, 1,2,3-triazole-4-carboxamide, 1,2,4-triazole-3-carboxamide, propargylglycine, Asp, Glu, or Cys;
wherein AA₅ is D-allo-Ile, or any other D- or L-amino acid with hydrophobic side chain;
wherein AA₆ is Ile, Val, Leu, Chg, or other amino acid with bulky hydrophobic side chain;
wherein AA₇ is Ser, Thr, allo-Thr, or other amino acid with beta hydroxy group;
wherein Ring is the teixobactin ring.

11. The composition of claim 10, wherein the teixobactin ring is conjugated to the vancomycin via a sidechain of AA₄.

12. The composition of claim 4, wherein the teixobactin tail comprises the following structure: 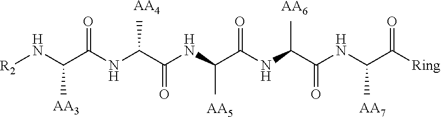
wherein R² is H, a methyl group, N-Me-d-Phe, an alkyl group C2 to C12, a benzyloxycarbonyl group, a benzoyl group, a halobenzoyl group, a biphenyl group, a halobiphenyl group, or an N-linkage that conjugates the vancomycin to the teixobactin tail;
wherein AA₃ is Ser, Thr, allo-Thr, or other amino acid with beta hydroxy group;
wherein AA₄ is D-Gln, any other D-amino acid, diamino propionic acid (Dap), 2,4-diamino butyric acid, Orn, Lys, 6-azidolysine, 2,7-diaminoheptanoic acid, 1,2,3-triazole-4-carboxamide, 1,2,4-triazole-3-carboxamide, propargylglycine, Asp, Glu, or Cys;
wherein AA₅ is D-allo-Ile, or any other D- or L-amino acid with hydrophobic side chain;
wherein AA₆ is Ile, Val, Leu, Chg, or other amino acid with bulky hydrophobic side chain;
wherein AA₇ is Ser, Thr, allo-Thr, or other amino acid with beta hydroxy group;
wherein Ring is the teixobactin ring.

13. The composition of claim 4, wherein the teixobactin tail comprises the following structure: 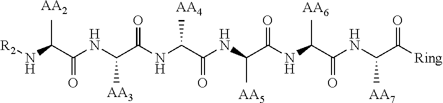
wherein R² is H, a methyl group, N-Me-d-Phe, an alkyl group C2 to C12, a benzyloxycarbonyl group, a benzoyl group, a halobenzoyl group, a biphenyl group, a halobiphenyl group, or an N-linkage that conjugates the vancomycin to the teixobactin tail;
wherein AA₂ is Ile, Val, Leu, Chg, or other amino acid with bulky hydrophobic side chain;
wherein AA₃ is Ser, Thr, allo-Thr, or other amino acid with beta hydroxy group;
wherein AA₄ is D-Gln, any other D-amino acid, diamino propionic acid (Dap), 2,4-diamino butyric acid, Orn, Lys, 6-azidolysine, 2,7-diaminoheptanoic acid, 1,2,3-triazole-4-carboxamide, 1,2,4-triazole-3-carboxamide, propargylglycine, Asp, Glu, or Cys;
wherein AAs is D-allo-Ile, or any other D- or L-amino acid with hydrophobic side chain;
wherein AA₆ is Ile, Val, Leu, Chg, or other amino acid with bulky hydrophobic side chain;
wherein AA₇ is Ser, Thr, allo-Thr, or other amino acid with beta hydroxy group;
wherein Ring is the teixobactin ring.

14. The composition of claim 4, wherein the teixobactin tail comprises the following structure: 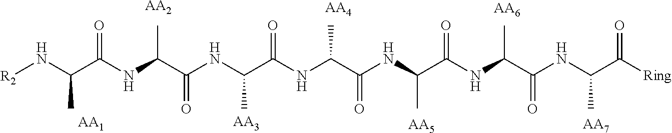
wherein R² is H, a methyl group, N-Me-d-Phe, an alkyl group C2 to C12, a benzyloxycarbonyl group, a benzoyl group, a halobenzoyl group, a biphenyl group, a halobiphenyl group, or an N-linkage that conjugates the vancomycin to the teixobactin tail;
wherein AA₁ is N-Me-D-Phe, Phe, or any other hydrophobic amino acid;
wherein AA₂ is Ile, Val, Leu, Chg, or other amino acid with bulky hydrophobic side chain;
wherein AA₃ is Ser, Thr, allo-Thr, or other amino acid with beta hydroxy group;
wherein AA₄ is D-Gln, any other D-amino acid, diamino propionic acid (Dap), 2,4-diamino butyric acid, Orn, Lys, 6-azidolysine, 2,7-diaminoheptanoic acid, 1,2,3-triazole-4-carboxamide, 1,2,4-triazole-3-carboxamide, propargylglycine, Asp, Glu, or Cys;
wherein AA₅ is D-allo-Ile, or any other D- or L-amino acid with hydrophobic side chain;
wherein AA₆ is Ile, Val, Leu, Chg, or other amino acid with bulky hydrophobic side chain;
wherein AA₇ is Ser, Thr, allo-Thr, or other amino acid with beta hydroxy group;
wherein Ring is the teixobactin ring.

15. The composition of any one of claim 1, wherein the vancomycin comprises the following structure: 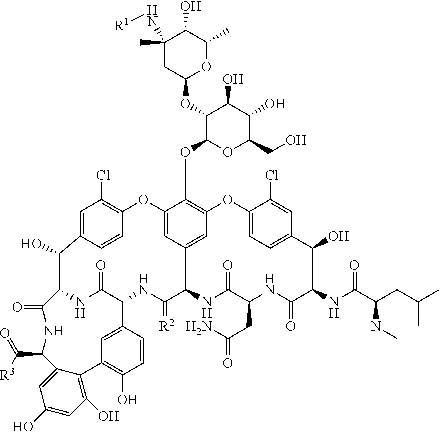
wherein R¹ is H, a benzyloxycarbonyl group, a benzoyl group, a halobenzoyl group, a biphenyl group, a halobiphenyl group, or an N-linkage that conjugates the vancomycin to the teixobactin ring;
wherein R² is O, H, or N that provides an N-linkage that conjugates the vancomycin to the teixobactin ring;
wherein R³ is H, a benzyloxycarbonyl group, a benzoyl group, a halobenzoyl group, a biphenyl group, a halobiphenyl group, or an N-linkage that conjugates the vancomycin to the teixobactin ring;
wherein R⁴ is H, a benzyloxycarbonyl group, a benzoyl group, a halobenzoyl group, a biphenyl group, a halobiphenyl group, or an N-linkage that conjugates the vancomycin to the teixobactin ring.

16. The compound of claim 15, wherein one or more hydroxyl groups is substituted with a sugar moiety.

17. The compound of claim 1 further comprising a linker between the teixobactin ring and the vancomycin to provide conjugation.

18. The compound of claim 1, wherein the linker comprises is formulated as a medicament.
